SEQUENCE LISTING (125 sequences)

SEQ ID NO: 1 - DNA, 375 bp, Vicugna pacos
caggtgcagctgcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcacaccaccatcaccatcac

SEQ ID NO: 2 - DNA, 357 bp, Vicugna pacos
caggtgcagctgcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctca

SEQ ID NO: 3 - protein, 125 aa, Vicugna pacos
QVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSHHHHHH

SEQ ID NO: 4 - protein, 119 aa, Vicugna pacos
QVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSS

SEQ ID NO: 5 - DNA, 390 bp, Vicugna pacos
caggtgcagctgcaggagtctggaggaggattggtgcaggctgggggctctctgagactctcctgtgcagcctctggacgcaccttcagtagagatgccatgggctggttccgccaggctccagggaaggagcgtgagtttgtagcaggtattagctggagtggtggtagcacatactatgcagactccgtgaagggccgattcaccatctccagggacggcgccaagaacacggtaaatctgcaaatgaacagcctgaaacctgaggacacggccgtttattactgtgcagcatcgtcgatttatgggagtgcggtagtagatgggctgtatgactactggggccaggggacccaggtcaccgtctcctcacaccaccatcaccatcac

SEQ ID NO: 6 - DNA, 372 bp, Vicugna pacos
caggtgcagctgcaggagtctggaggaggattggtgcaggctgggggctctctgagactctcctgtgcagcctctggacgcaccttcagtagagatgccatgggctggttccgccaggctccagggaaggagcgtgagtttgtagcaggtattagctggagtggtggtagcacatactatgcagactccgtgaagggccgattcaccatctccagggacggcgccaagaacacggtaaatctgcaaatgaacagcctgaaacctgaggacacggccgtttattactgtgcagcatcgtcgatttatgggagtgcggtagtagatgggctgtatgactactggggccaggggacccaggtcaccgtctcctca

SEQ ID NO: 7 - protein, 130 aa, Vicugna pacos
QVQLQESGGGLVQAGGSLRLSCAASGRTFSRDAMGWFRQAPGKEREFVAGISWSGGSTYYADSVKGRFTISRDGAKNTVNLQMNSLKPEDTAVYYCAASSIYGSAVVDGLYDYWGQGTQVTVSSHHHHHH

SEQ ID NO: 8 - protein, 124 aa, Vicugna pacos
QVQLQESGGGLVQAGGSLRLSCAASGRTFSRDAMGWFRQAPGKEREFVAGISWSGGSTYYADSVKGRFTISRDGAKNTVNLQMNSLKPEDTAVYYCAASSIYGSAVVDGLYDYWGQGTQVTVSS

SEQ ID NO: 9 - DNA, 20 bp, artificial sequence
  Primer
cccatgaagaccttcacctc

SEQ ID NO: 10 - DNA, 20 bp, artificial sequence
  Primer
catccctggaacactccact

SEQ ID NO: 11 - DNA, 20 bp, artificial sequence
  Primer
actccttgattggtggaagc

SEQ ID NO: 12 - DNA, 20 bp, artificial sequence
  Primer
caaaatggcacagacattgg

SEQ ID NO: 13 - DNA, 20 bp, artificial sequence
  Primer
tcctttccccaaatatcacg

SEQ ID NO: 14 - DNA, 20 bp, artificial sequence
  Primer
cagccatccctaccattcat

SEQ ID NO: 15 - DNA, 20 bp, artificial sequence
  Primer
gtgcccacgtcaaggagtat

SEQ ID NO: 16 - DNA, 20 bp, artificial sequence
  Primer
agcaagcaatgacagggaag

SEQ ID NO: 17 - DNA, 20 bp, artificial sequence
  Primer
gtcttctggagtaccatagc

SEQ ID NO: 18 - DNA, 20 bp, artificial sequence
  Primer
gtcagatacctgacaacagg

SEQ ID NO: 19 - DNA, 20 bp, artificial sequence
  Primer
tctgagtcctcgctcaagtg

SEQ ID NO: 20 - DNA, 20 bp, artificial sequence
  Primer
ccttgggaagatggtggtta

SEQ ID NO: 21 - DNA, 20 bp, artificial sequence
  Primer
tcaacaaaagagctgccaaa

SEQ ID NO: 22 - DNA, 20 bp, artificial sequence
  Primer
gcagaggccagaagagagaa

SEQ ID NO: 23 - DNA, 20 bp, artificial sequence
  Primer
gaaagaccctgaccatcact

SEQ ID NO: 24 - DNA, 20 bp, artificial sequence
  Primer
ccttctctgcagacagagac

SEQ ID NO: 25 - DNA, 20 bp, artificial sequence
  Primer
gtgtggatccaaagcaatac

SEQ ID NO: 26 - DNA, 20 bp, artificial sequence
  Primer
gtctgctcattcatgacaag

SEQ ID NO: 27 - DNA, 20 bp, artificial sequence
  Primer
gcactgtgtgccgataaaga

SEQ ID NO: 28 - DNA, 20 bp, artificial sequence
  Primer
tacctccgggaaatgacatc

SEQ ID NO: 29 - DNA, 20 bp, artificial sequence
  Primer
tgaatcagctggcttttgtg

SEQ ID NO: 30 - DNA, 20 bp, artificial sequence
  Primer
gtggatagctcggtggtgtt

SEQ ID NO: 31 - DNA, 20 bp, artificial sequence
  Primer
caggctgaacttcgaaacag

SEQ ID NO: 32 - DNA, 20 bp, artificial sequence
  Primer
cagctacgaaaacccaatca

SEQ ID NO: 33 - DNA, 21 bp, artificial sequence
  Primer
gcttctggtcgatgtcatgag

SEQ ID NO: 34 - DNA, 20 bp, artificial sequence
  Primer
tccaccaggagatgttgaac

SEQ ID NO: 35 - DNA, 20 bp, artificial sequence
  Primer
gcatgtggtccttccaactt

SEQ ID NO: 36 - DNA, 20 bp, artificial sequence
  Primer
gatcctcagccacaaccttc

SEQ ID NO: 37 - DNA, 20 bp, artificial sequence
  Primer
tgacttgggtccttgtcctc

SEQ ID NO: 38 - DNA, 20 bp, artificial sequence
  Primer
aaggaagccaccaatgacac

SEQ ID NO: 39 - DNA, 20 bp, artificial sequence
  Primer
acttcgcaggagaactggag

SEQ ID NO: 40 - DNA, 20 bp, artificial sequence
  Primer
aagaagctgttgggaggaca

SEQ ID NO: 41 - DNA, 20 bp, artificial sequence
  Primer
caggctgctgtaacgatgaa

SEQ ID NO: 42 - DNA, 20 bp, artificial sequence
  Primer
aatgctttctccgctctgaa

SEQ ID NO: 43 - DNA, 20 bp, artificial sequence
  Primer
gaaagcatacctggctggac

SEQ ID NO: 44 - DNA, 20 bp, artificial sequence
  Primer
acaaaagagccgtacctgga

SEQ ID NO: 45 - DNA, 20 bp, artificial sequence
  Primer
tttcaaaaggaaggggacaa

SEQ ID NO: 46 - DNA, 20 bp, artificial sequence
  Primer
ccacctagaaaaccctgctg

SEQ ID NO: 47 - DNA, 21 bp, artificial sequence
  Primer
actcaatacacactgcaggtg

SEQ ID NO: 48 - DNA, 20 bp, artificial sequence
  Primer
ggactttaagggttacttgg

SEQ ID NO: 49 - DNA, 20 bp, artificial sequence
  Primer
gtctcctgcctccactttct

SEQ ID NO: 50 - DNA, 20 bp, artificial sequence
  Primer
ctaagggcagaggggctatt

SEQ ID NO: 51 - DNA, 20 bp, artificial sequence
  Primer
ccttcacagagcaatgactc

SEQ ID NO: 52 - DNA, 21 bp, artificial sequence
  Primer
gtctactcccaggttctcttc

SEQ ID NO: 53 - DNA, 20 bp, artificial sequence
  Primer
cgttgccattggaatagaga

SEQ ID NO: 54 - DNA, 20 bp, artificial sequence
  Primer
tggcaaagagtcaaaactgg

SEQ ID NO: 55 - DNA, 20 bp, artificial sequence
  Primer
caccattagtctgggcgtct

SEQ ID NO: 56 - DNA, 20 bp, artificial sequence
  Primer
gatgcggaagtagcaaaagc

SEQ ID NO: 57 - DNA, 20 bp, artificial sequence
  Primer
cttttagcggcacgaacgat

SEQ ID NO: 58 - DNA, 20 bp, artificial sequence
  Primer
aagaacagcggtgcaggtaa

SEQ ID NO: 59 - DNA, 20 bp, artificial sequence
  Primer
tgacatgcccaagactcaga

SEQ ID NO: 60 - DNA, 20 bp, artificial sequence
  Primer
aggttgctcaagcagcaaag

SEQ ID NO: 61 - DNA, 20 bp, artificial sequence
  Primer
ccggtactactgctgctcct

SEQ ID NO: 62 - DNA, 20 bp, artificial sequence
  Primer
cacacaccgagctgtgagat

SEQ ID NO: 63 - DNA, 20 bp, artificial sequence
  Primer
ctcagttcatccacggcata

SEQ ID NO: 64 - DNA, 20 bp, artificial sequence
  Primer
caaggctcaccatcatcgta

SEQ ID NO: 65 - DNA, 20 bp, artificial sequence
  Primer
tctcctgggcaagtgtagga

SEQ ID NO: 66 - DNA, 20 bp, artificial sequence
  Primer
gcctgtgcagagtgaacaaa

SEQ ID NO: 67 - DNA, 20 bp, artificial sequence
  Primer
ctccacagcgcttctattcc

SEQ ID NO: 68 - DNA, 20 bp, artificial sequence
  Primer
cttcttcttggggtcagcac

SEQ ID NO: 69 - DNA, 20 bp, artificial sequence
  Primer
ctgggcaagaaatctgatga

SEQ ID NO: 70 - DNA, 20 bp, artificial sequence
  Primer
tggttgtggcaggaaagata

SEQ ID NO: 71 - DNA, 20 bp, artificial sequence
  Primer
ggatgttgacagcaagagca

SEQ ID NO: 72 - DNA, 20 bp, artificial sequence
  Primer
ctcatcttcaccccggttag

SEQ ID NO: 73 - DNA, 20 bp, artificial sequence
  Primer
ccaaggagacggaatacagg

SEQ ID NO: 74 - DNA, 20 bp, artificial sequence
  Primer
tctctgtggagctgaagcaa

SEQ ID NO: 75 - DNA, 21 bp, artificial sequence
  Primer
tcatagccacactcaagaatg

SEQ ID NO: 76 - DNA, 21 bp, artificial sequence
  Primer
aagcagaactgaactaccatc

SEQ ID NO: 77 - DNA, 20 bp, artificial sequence
  Primer
tctacgcagtgcttctttgc

SEQ ID NO: 78 - DNA, 20 bp, artificial sequence
  Primer
ccacttctgtgtggggtcta

SEQ ID NO: 79 - DNA, 20 bp, artificial sequence
  Primer
gcagatggctcatgtctgaa

SEQ ID NO: 80 - DNA, 20 bp, artificial sequence
  Primer
ctctgggaagctgggtgtag

SEQ ID NO: 81 - DNA, 20 bp, artificial sequence
  Primer
tcacctgagctttgatgtcg

SEQ ID NO: 82 - DNA, 20 bp, artificial sequence
  Primer
ttatggttaccctcccgttg

SEQ ID NO: 83 - DNA, 20 bp, artificial sequence
  Primer
gcttggcttatggactgagg

SEQ ID NO: 84 - DNA, 20 bp, artificial sequence
  Primer
cttgtccttgtggctgtgaa

SEQ ID NO: 85 - DNA, 20 bp, artificial sequence
  Primer
gcctcctgctcatagctacc

SEQ ID NO: 86 - DNA, 20 bp, artificial sequence
  Primer
gggtcagcacagatctcctt

SEQ ID NO: 87 - DNA, 20 bp, artificial sequence
  Primer
atgtccagctttgtgggttc

SEQ ID NO: 88 - DNA, 20 bp, artificial sequence
  Primer
aggtcaggttccgcagataa

SEQ ID NO: 89 - DNA, 20 bp, artificial sequence
  Primer
cccacttcctgctgtttctc

SEQ ID NO: 90 - DNA, 20 bp, artificial sequence
  Primer
gagcaaggacgcttctcagt

SEQ ID NO: 91 - DNA, 21 bp, artificial sequence
  Primer
ccttcgcgattatcagaatcc

SEQ ID NO: 92 - DNA, 20 bp, artificial sequence
  Primer
tacttatggtggacccagca

SEQ ID NO: 93 - DNA, 20 bp, artificial sequence
  Primer
ccagatcacacatgcaacag

SEQ ID NO: 94 - DNA, 25 bp, artificial sequence
  Primer
ctataaaaataaacacttagagcca

SEQ ID NO: 95 - DNA, 21 bp, artificial sequence
  Primer
cggaagattccacgccaattc

SEQ ID NO: 96 - DNA, 21 bp, artificial sequence
  Primer
ggtgaggaacgtgtcctgaag

SEQ ID NO: 97 - DNA, 20 bp, artificial sequence
  Primer
atcgaacccagagtggaatg

SEQ ID NO: 98 - DNA, 20 bp, artificial sequence
  Primer
gctagaaaccccagcatgag

SEQ ID NO: 99 - DNA, 25 bp, artificial sequence
  Primer
cactcacctgctgctactcattcac

SEQ ID NO: 100 - DNA, 24 bp, artificial sequence
  Primer
ggattcacagagagggaaaaatgg

SEQ ID NO: 101 - DNA, 20 bp, artificial sequence
  Primer
gacaaagaagggcatggaag

SEQ ID NO: 102 - DNA, 20 bp, artificial sequence
  Primer
cattccttaggcgtgaccat

SEQ ID NO: 103 - DNA, 20 bp, artificial sequence
  Primer
gcaaatggagccgtctgtgc

SEQ ID NO: 104 - DNA, 20 bp, artificial sequence
  Primer
ctcgtggatctccgtgacac

SEQ ID NO: 105 - DNA, 20 bp, artificial sequence
  Primer
acgggaaactgcttgatgtc

SEQ ID NO: 106 - DNA, 20 bp, artificial sequence
  Primer
actcagcgtcatgttgtcca

SEQ ID NO: 107 - DNA, 20 bp, artificial sequence
  Primer
gagcatgaatgaagtgtccg

SEQ ID NO: 108 - DNA, 20 bp, artificial sequence
  Primer
tgctgaagttgtcgtcacac

SEQ ID NO: 109 - DNA, 20 bp, artificial sequence
  Primer
ctggctgtttgctacgtgaa

SEQ ID NO: 110 - DNA, 20 bp, artificial sequence
  Primer
catgaaacttgcctcgtgtg

SEQ ID NO: 111 - DNA, 780 bp, Vicugna pacos
caggtgcagcttcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcaagcccatctacacctcccacaccatcaccatccacaccaccggcaagtcaggtgcagctgcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcacaccaccatcaccatcac

SEQ ID NO: 112 - protein, 260 aa, Vicugna pacos
QVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSSPSTPPTPSPSTPPASQVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSHHHHHH

SEQ ID NO: 113 - DNA, 777 bp, Vicugna pacos
caggtgcagcttcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcaggcggaggcggtagtggcggaggtggatctggaggcggcggtagtcaggtgcagctgcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcacaccaccatcaccatcac

SEQ ID NO: 114 - protein, 259 aa, Vicugna pacos
QVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSGGGGSGGGGSGGGGSQVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSHHHHHH

SEQ ID NO: 115 - DNA, 783 bp, Vicugna pacos
caggtgcagcttcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcagcgcaccacagcgaagaccccagctccaaagctcccaaagctccaatggcacaggtgcagctgcaggagtctggaggaggcttggtgcagcctggggggtctctgagactctcctgtgcagcctctggaaacatcttcagtatcaatgccatcggctggtaccgccaggctccagggaagcagcgcgagttggtcgcaactattactcttagtggtagcacaaactatgcagactccgtgaagggccgattctccatctccagagacaacgccaagaacacggtgtatctgcaaatgaacagcctgaaacctgaggacacggccgtctattactgtaatgctaacacctatagcgactctgacgtttatggctactggggccaggggacccaggtcaccgtctcctcacaccaccatcaccatcac

SEQ ID NO: 116 - protein, 261 aa, Vicugna pacos
QVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSAHHSEDPSSKAPKAPMAQVQLQESGGGLVQPGGSLRLSCAASGNIFSINAIGWYRQAPGKQRELVATITLSGSTNYADSVKGRFSISRDNAKNTVYLQMNSLKPEDTAVYYCNANTYSDSDVYGYWGQGTQVTVSSHHHHHH

SEQ ID NO: 117 - DNA, 41 bp, Artificial Sequence
  Primer
ccggccatggcccaggtgcagcttcaggagtctggaggagg

SEQ ID NO: 118 - DNA, 88 bp, Artificial Sequence
  Primer
tgattcctgcagctgcacctgactaccgccgcctccagatccacctccgccactaccgcctccgcctgaggagacggtgacctgggtc

SEQ ID NO: 119 - DNA, 94 bp, Artificial Sequence
  Primer
tgattcctgcagctgcacctgtgccattggagctttgggagctttggagctggggtcttcgctgtggtgcgctgaggagacggtgacctgggtc

SEQ ID NO: 120 - DNA, 91 bp, Artificial Sequence
  Primer
tgattcctgcagctgcacctgacttgccggtggtgtggatggtgatggtgtgggaggtgtagatgggcttgaggagacggtgacctgggtc

SEQ ID NO: 121 - protein, 15 aa, Artificial Sequence
  Linker
GGGGSGGGGSGGGGS

SEQ ID NO: 122 - protein, 17 aa, Artificial Sequence
  Linker
AHHSEDPSSKAPKAPMA

SEQ ID NO: 123 - protein, 16 aa, Artificial Sequence
  Linker
SPSTPPTPSPSTPPAS

SEQ ID NO: 124 - DNA, 40 bp, Artificial Sequence
  Primer
attgaattctattagtggtggtggtggtggtgctcgagtg

SEQ ID NO: 125 - DNA, 33 bp, Artificial Sequence
  Primer
ttaactgcagatggccgaagagggcggcagcct

(19) United States
(12) Patent Application Publication
(10) Pub. No.: US 2011/0262348 A1
(43) Pub. Date: Oct. 27, 2011

(54) Selective targeting of intratumoral cells

(75) Inventors: Kiavash Movahedi, Frankfurt am Main (DE); Damya Laoui, Limal (BE); Steve Schoonooghe, Kessel-Lo (BE); Geert Raes, Sint-Genesius-Rode (BE); Patrick De Baetselier, Berchem (BE); Jo Van Ginderachter, Ninove (BE); Nick Devoogdt, Zemst (BE); Tony Lahoutte, Ganshoren (BE)

(73) Assignees: Vrije Universiteit Brussel, Brussel (BE); VIB VZW, Gent (BE)

(21) Appl. No.: 13/065,794

(22) Filed: Mar. 29, 2011

Related U.S. Application Data
(60) Provisional application No. 61/341,356, filed on Mar. 29, 2010.

(51) Int. Cl. A61K 39/395; A61P 35/00; A61K 51/10; C07K 16/28; C12N 11/02

(52) U.S. Cl. 424/1.49; 530/387.3; 530/391.7; 530/391.3; 435/177; 424/133.1; 424/178.1

(57) ABSTRACT

The present invention relates to the field of tumor growth and biology. The invention relates to activities and characteristics of tumor-associated macrophages, and uses of such for the diagnosis and treatment of cancer and tumor growth.

### CROSS-REFERENCE TO RELATED APPLICATION

This application claims the benefit of U.S. Provisional Patent Application Ser. No. 61/341,356, filed Mar. 29, 2010, the disclosure of which is hereby incorporated herein in its entirety by this reference.

### FIELD OF THE INVENTION

The present invention relates to the field of tumor growth and biology. The invention relates to activities and characteristics of tumor-associated macrophages (TAMs), and uses of such for the diagnosis and treatment of cancer and tumor growth.

### BACKGROUND

Myeloid cells are frequently found to infiltrate tumors and have been linked to diverse tumor-promoting activities.⁽¹⁾ In particular, tumor-associated macrophages (TAMs) are an important component of the tumor stroma, both in murine models and human patients.⁽²⁾ TAMs can promote tumor-growth by affecting angiogenesis, immune suppression and invasion and metastasis.^{(2,3)}

Tissue-resident macrophages can be maintained through local proliferation or differentiation in situ from circulating monocytic precursors.⁽⁵⁾ Importantly, discrete subsets of blood monocytes have been described. Mouse monocytes can be classified as Ly6C^{low}CX₃CR1^{hi}(CCR2⁻CD62L⁻) or Ly6C^{hi}CX₃CR1^{low}(CCR2⁺CD62L⁺) and are shown to have distinct functions and migration patterns.⁽⁶⁾

Macrophages are plastic cells that can adopt different phenotypes depending on the immune context. Microenvironmental stimuli can drive a macrophage either towards a “classical” (M1) or an “alternative” (M2) activation state, two extremes in a spectrum.⁽⁷⁾ M1 macrophages are typically characterized by the expression of pro-inflammatory cytokines, inducible nitric oxide synthase 2 (Nos2) and MHC Class II molecules. M2 macrophages, have a decreased level of the aforementioned molecules and are identified by their signature-expression of a variety of markers, including arginase-1 and mannose and scavenger receptors. It has been suggested that TAMs display a M2-like phenotype.⁽⁸⁾

Despite the presence of TAM in tumor infiltrate and their potential to produce angiogenic factors, their role in tumor growth and development remains unclear. There remains a need to discover and understand the complexities of the tumor-infiltrating myeloid cell compartment in view of the selective treatment of tumor growth.

### SUMMARY OF THE INVENTION

The present invention is based on the inventor's surprising finding of the existence of molecularly and functionally distinct TAM subsets, located in different intratumoral regions and the unraveling of Ly6C^{hi} monocytes as their precursors. In particular, molecular markers for discriminating between these different TAM subsets, and accordingly, between these different intratumoral microenvironments (hypoxic versus normoxic zones), form the basis of the present invention. The present invention relates to the use of these molecular markers for specifically targeting the M1/M2-like or hypoxic/perivascular TAM subsets or their precursors, or, in a preferred embodiment, for selectively targeting the hypoxic/perivascular cells inside a tumor. The invention further relates to combinatorial strategies for optimally “re-educating” the TAM compartment and reverting its tumor-promoting activities. The invention also relates to diagnostic/prognostic applications based on the existence of distinct TAM subsets and their corresponding molecular markers.

Objects of the present invention will be clear from the description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: TS/A tumors are infiltrated by distinct granulocyte and monocyte/macrophage subsets. (A) Single-cell suspensions of 11-day-old tumors were stained for the indicated markers. On gated CD11b+ cells, Ly6C was plotted vs. MHC II, demonstrating at least 7 distinct subsets. For each subset, forward scatter (FSC) vs. side scatter (SSC) plots are shown. (B) Staining single-cell suspensions from 7, 11, 14- and 21-day-old tumors. Plots are gated on CD11b+ cells. Accompanying mean tumor diameters±SEM are indicated. n=3 experiments. (C) The expression of the indicated markers was assessed on cells present in gates 1-4, as shown in panel A. All markers were analyzed using antibody staining, except for CX3CR1, for which tumors were grown in CX3CR1GFP/+ mice. Shaded histograms are isotype controls or, for CX3CR1, autofluorescence in WT mice.

FIG. 2: Infiltration of latex-labeled monocytes in tumors and kinetics of BrdU incorporation in the distinct TAM subsets. (A) 6-day-old tumors were collected from control mice or mice in which the Ly6Clow or Ly6Chi monocytes were labeled. Plots are gated on CD11b+ cells. n=3 experiments. (B1) 6-, 12- or 19-day-old tumors were collected from untreated mice (control) or mice in which the Ly6Chi monocytes were labeled (latex injected). Plots are gated on CD11b+ cells. (B2) Ly6C vs. MHC II plots of tumor single-cell suspensions from latex injected mice at 6, 12 or 19 days p.i, either gated on the total CD11b+ population or on the latex+CD11b+ population. n=3 experiments. (C) 2 weeks tumor-bearing mice were left untreated (0 hour) or continuously given BrdU for the indicated time, after which BrdU incorporation in tumor cells was measured. C1 shows how BrdU+ cells were gated in the different TAM subsets. n=2 BrdU-kinetic experiments (D) The intracellular expression of Ki67 was assessed via flow cytometry. Shaded histograms are isotype controls. n>3.

FIG. 3: Arginase, TNFα, and iNOS protein expression in MHC IIhi and MHC IIlow TAMs. (A) Arginase enzymatic activity (mU) was measured in lysates of sorted TAMs. Values are the mean±SEM of 3 experiments. *p<0.05 (B) TNFα production by TAMs was measured using intracellular FACS. Bar diagrams represent the mean percentage TNFα+TAMs±SEM from 3 experiments. *p<0.05 (C) TAMs were left untreated or stimulated with IFNγ, LPS or LPS+IFNγ for 12 hours. Subsequently, iNOS expression was evaluated using intracellular FACS. The percentage iNOS+ cells is shown as normalized ΔMFI (see Materials & Methods). n=2 experiments.

FIG. 4: MHC IIlow TAMs are enriched in hypoxic regions, while MHC IIhi TAMs are mainly normoxic. (A) 3 weeks tumor-bearing mice were injected with pimonidazole (HP-1). Frozen tumor sections were stained with MECA32 and anti-HP-1 antibodies and DAPI. (B) Frozen tumor sections from HP-1 injected mice were stained for CD11b, MHC II, HP-1 adducts and DAPI. (C) Assessment of HP-1 adducts in the distinct tumor myeloid subsets using FACS. n=4 experiments.

FIG. 5: Differential functions of TAM subsets. (A) Sorted TAMs were grafted on the developing chorioallantoic membrane from fertilized chicken eggs. BSA and rhVEGF grafting were used as negative and positive controls, respectively. At day 13, the number of vessels growing towards the implants was quantified. Values are the mean number of implant-directed vessels±SEM of 8 individual eggs/condition of 2 experiments. *p<0.05; **p<0.01. (B) Sorted TAM subsets or splenic Balb/c cDCs were cultured in the presence of purified C57BL/6 CD4+ or CD8+ T cells and T-cell proliferation was assessed. Graphs represent the average level of 3H-thymidine incorporation, expressed as Counts Per Minute (CPM), =SEM. n=3 experiments. (C) Sorted TAM subsets or splenic Balb/c cDC were added to naive Balb/c splenocytes. Cocultures were stimulated with anti-CD3 and proliferation was assessed. n=3 experiments. (D) TAM subsets and Balb/c splenocytes were cultured at a 1:4 ratio and treated with anti-CD3 with or without the indicated inhibitors. Values represent the mean±SD of the relative percentage suppression taken over 3 experiments. *p<0.05

FIG. 6: Identifying Ly6C^{hi} and Ly6C^{low} monocytes in tumors. 11-day-old tumors were collected from CX₃CR1^{GFP/+} reporter mice. Within the gated CD11b⁺ population, Ly6C^{hi}MHC II⁻ and Ly6C^{low}MHC II⁻ cells were subgated and their respective CX₃CR1 vs. CCR3 plots are shown. Ly6C^{hi}MHC II⁻ cells were CCR3⁻CX₃CR1^{low} (Gate 1). Ly6C^{low}MHC II⁻ cells could be subdivided in CCR3⁻CX₃CR1^{low} (Gate 2), CCR3⁻CX₃CR1^{hi} (Gate 3) and CCR3⁺CX₃CR1⁻ cells (Gate E, comprising of eosinophils). Forward vs. Side Scatter plots for the distinct gates are shown in the bottom panel. Similar results were seen at different time points of tumor growth. For the indicated time point, results are representative of 3 independent experiments.

FIG. 7: Purities of sorted cell populations. Representative plots are shown of the FACS sorted cell populations that were used throughout the study. (A) MHC IIhi TAMs and MHC IIlow TAMs (B) CD11c+MHC IIhiB220-Ly6C-splenic cDCs.

FIG. 8: Latex bead uptake by TAM subsets in vivo and in vitro. (A) 3 weeks tumor-bearing mice were injected iv with fluorescent latex beads and 2 hours later, tumors were collected to assess latex uptake by the CD11b+ population. The depicted SSC vs. latex plot is on gated CD11b+ cells and shows how latex+ cells are gated. The percentage of Ly6Chi monocytes, Ly6Cint TAMs, MHC IIhi TAMs and MHC IIlow TAMs within the total CD11b+ gate or CD11b+Latex+ gate is depicted for 5 individual groups of tumors from three independent experiments. (B) Tumor single cell suspensions were cultured in vitro, at 4° C. or 37° C., in the absence (control) or presence of latex beads for 40 minutes. Latex+ cells within the CD11b+ population were gated and their percentages are given. The percentage of the distinct monocyte/TAM subsets within the total CD11b+ gate or CD11b+Latex+ gate is depicted for 5 individual groups of tumors from three independent experiments for cells cultured at 37° C.

FIG. 9: DQ-OVA processing by TAM subsets. TAMs were allowed to phagocytose and process DQ-OVA for 15 minutes at 0° C. or 37° C. Free DQ-OVA was subsequently removed from the culture medium and TAMs were given an additional 15, 30 and 60 minutes to process internalized DQ-OVA. DQ-OVA processing results in the formation of fluorescent peptides and fluorescence intensities for the gated TAM subsets are shown in histogram plots. Values are the mean percentage cells within the indicated gate±SEM from three independent experiments. p-values were calculated for these means between MHC II^{hi} vs. MHC II^{low} TAMs for each time point. *p<0.05

FIG. 10: Schematic summary.

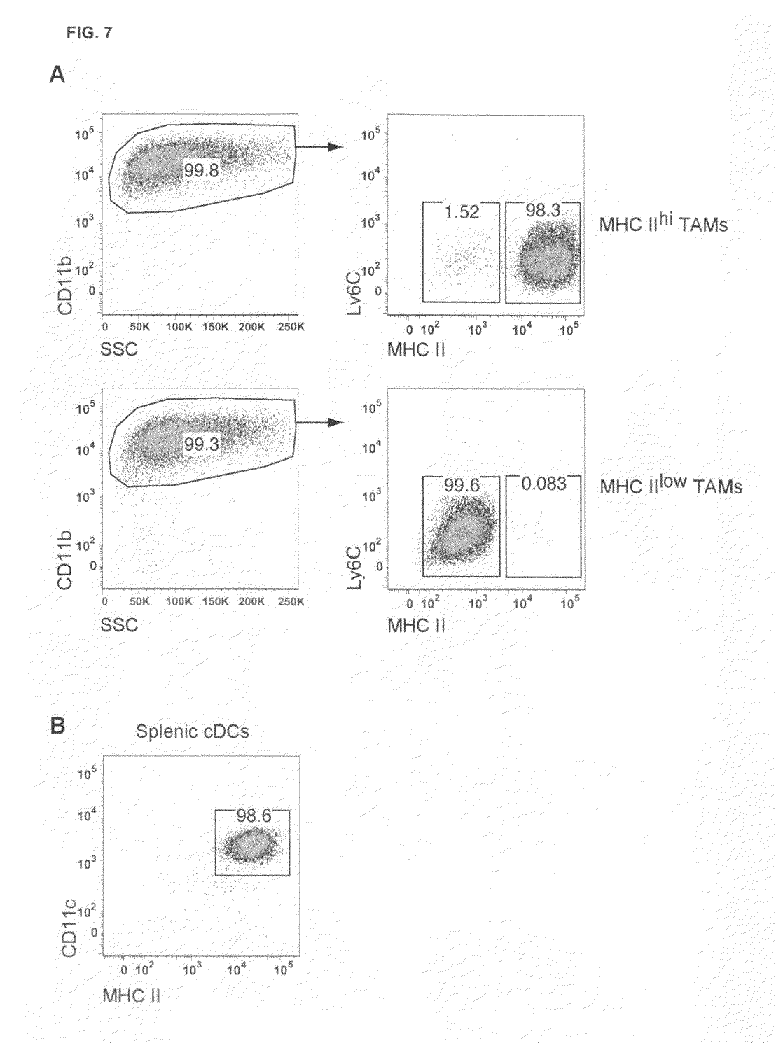

FIG. 11: Biodistribution MMR Nb in naïve and knockout mice.

FIG. 12: Uptake experiments of MMR Nb in TS/A tumor-bearing mice.

FIG. 13: TAM subsets in the Lewis Lung Carcinoma (LLC) model and in the mammary carcinoma model 4T1.

FIG. 14. MMR expression on distinct cell types present in TS/A tumor suspensions. Single cell suspensions were made from TS/A tumors and MMR expression was evaluated on the indicated cell populations using an anti-MMR monoclonal antibody. Shaded histograms represent isotype control.

FIG. 15. Anti-MMR clone 1 differentially labels TAM subsets in TS/A tumor sections. TS/A tumors were collected from 3 weeks tumor-bearing mice and frozen sections were triple-stained for MMR (red), MHC II (green) and CD11b (blue).

FIG. 16. anti-MMR Nb differentially binds to TAM subsets in tumor single cell suspensions. (A) Single-cell suspensions of 21-day-old TS/A tumors were stained with the indicated markers. anti-BCII10 Nb served as negative control. (B) Staining of anti-MMR Nb clone 1 was examined on the gated myeloid subsets. Shaded histograms represents staining with anti-BCII10 Nb.

FIG. 17. Coronal and sagittal views of fused Pinhole SPECT and Micro-CT images of naive WT or MMR^{-/-} mice 1 hour after injection with ^{99m}Tc labeled anti-MMR Nb clone 1. (A) In WT mice anti-MMR Nb shows kidney/bladder elimination and uptake in several organs. (B) In MMR^{-/-} mice anti-MMR Nb shows primarily kidney/bladder elimination.

FIG. 18. Coronal and transverse views of fused Pinhole SPECT and Micro-CT images of WT TS/A tumor-bearing mice 3 hours after injection with ^{99m}Tc labeled cAbBCII10 or anti-MMR Nb.

FIG. 19. Coronal and transverse views of fused Pinhole SPECT and Micro-CT images of WT and MMR^{-/-} 3LL tumor-bearing mice 3 h after injection with ^{99m}Tc labeled cAbBCII10 or anti-MMR Nb.

FIG. 20. Uptake values of ^{99m}Tc-labeled monovalent anti-MMR Nb clone 1 in TS/A tumor-bearing mice upon co-injection with an 80-fold excess of cold monovalent anti-MMR Nb, based on dissection at 3 hours post injection. Tracer uptake is expressed as injected activity per gram (% IA/g).

FIG. 21. Uptake values of ^{99m}Tc-labeled monovalent anti-MMR Nb clone 1 in TS/A tumor-bearing mice upon co-injection with a 20-fold excess of cold bivalent anti-MMR Nb, based on dissection at 3 hours post injection. Tracer uptake is expressed as injected activity per gram (% IA/g).

FIG. 22. The relative abundance of TAM subsets is different in fast growing 3LL-R versus slow growing 3LL-S tumors. 3×10⁶ cancer cells were injected in the flank and tumor volumes were measured at different time intervals. When tumors reached a volume of about 1000 mm³, tumor single cell suspensions were made and the presence of TAM subsets were assessed via FACS.

FIG. 23. MHC II^{hi} TAM are located outside of hypoxic regions in 3LL-R tumors. 3LL-R tumors were collected from 12-days tumor-bearing mice and frozen sections were double-stained for MHC II (green) and Hypoxyprobe (blue). Pictures are shown from three distinct regions within the same tumor.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g., “a” or “an,” “the,” this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Unless otherwise defined herein, scientific and technical terms and phrases used in connection with the present invention shall have the meanings that are commonly understood by those of ordinary skill in the art. Generally, nomenclatures used in connection with, and techniques of molecular and cellular biology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, for example, Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002).

As used herein, the terms “polypeptide,” “protein,” “peptide” are used interchangeably herein, and refer to a polymeric form of amino acids of any length, which can include coded and non-coded amino acids, chemically or biochemically modified or derivatized amino acids, and polypeptides having modified peptide backbones.

As used herein, the terms “nucleic acid molecule,” “polynucleotide,” “polynucleic acid,” “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

In a first aspect, the invention relates to a nanobody specifically recognizing a molecular marker of Table 1. In particular, the invention relates to a nanobody specifically binding to a macrophage mannose receptor. According to a specific embodiment, the nanobody of the invention specifically binds to the ectodomain of the macrophage mannose receptor.

The “macrophage mannose receptor” (MMR), as used herein, refers to a type 1 transmembrane protein, first identified in mammalian tissue macrophages and later in dendritic cells and a variety of endothelial and epithelial cells. Macrophages are central actors of the innate and adaptive immune responses. They are disseminated throughout most organs to protect against entry of infectious agents by internalizing and most of the time, killing them. Among the surface receptors present on macrophages, the mannose receptor recognizes a variety of molecular patterns generic to microorganisms. The MMR is composed of a single subunit with N- and O-linked glycosylations and consists of five domains: an N-terminal cysteine-rich region, which recognizes terminal sulfated sugar residues; a fibronectin type II domain with unclear function; a series of eight C-type, lectin-like carbohydrate recognition domains (CRDs) involved in Ca²⁺-dependent recognition of mannose, fucose, or N-acetylglucosamine residues on the envelop of pathogens or on endogenous glycoproteins with CRDs 4-8 showing affinity for ligands comparable with that of intact MR; a single transmembrane domain; and a 45 residue-long cytoplasmic tail that contains motifs critical for MR-mediated endocytosis and sorting in endosomes.⁽³⁴⁾

The macrophage mannose receptor as referred to in the present invention also includes homologues as wells as fragments of the full length MMR protein. Non-limiting examples of homologues of the MMR include the mouse MMR (synonyms: MRC1 or CD206; accession number nucleotide sequence: NM_{—}008625.2; accession number protein sequence: NP_{—}032651.2) or the human MMR (synonyms: Mrc1 or CD206; accession number nucleotide sequence: NM_{—}002438.2; accession number protein sequence: NP_{—}002429.1). The deduced amino acid sequence of mouse mannose receptor has an overall 82% homology with the human mannose receptor.

The “ectodomain” as used herein, refers to a fragment of the MMR containing an N-terminus that is cysteine-rich, followed by a fibronectin type II domain and eight carbohydrate recognition domains (CRDs). All of the eight CRDs are particularly well conserved, especially CRD4, which shows 92% homology with the equivalent region of the human protein. For example, the ectodomain of the mouse macrophage mannose receptor is defined as the AA 19-AA 1388 fragment of the corresponding full length mouse MMR amino acid sequence as defined in NP_{—}032651.2. Or, the ectodomain of the human macrophage mannose receptor is be defined as the AA 19-AA 1383 fragment of the corresponding full length mouse MMR amino acid sequence as defined in NP_{—}002429.1.

The present invention thus provides for nanobodies specifically recognizing a macrophage mannose receptor (as defined above). As used herein, the term “specifically recognizing” or “specifically binding to” or simply “specificity” refers to the ability of an immunoglobulin or an immunoglobulin fragment, such as a nanobody, to bind preferentially to one antigenic target versus a different antigenic target and does not necessarily imply high affinity.

A nanobody (Nb) is the smallest functional fragment or single variable domain (V_{H}H) of a naturally occurring single-chain antibody and is known to the person skilled in the art. They are derived from heavy chain only antibodies, seen in camelids.^{(26,27)} In the family of “camelids” immunoglobulins devoid of light polypeptide chains are found. “Camelids” comprise old world camelids (Camelus bactrianus and Camelus dromedarius) and new world camelids (for example, Lama paccos, Lama glama, Lama guanicoe and Lama vicugna). The single variable domain heavy chain antibody is herein designated as a Nanobody or a V_{H}H antibody. Nanobody™, Nanobodies™ and Nanoclone™ are trademarks of Ablynx Nev. (Belgium). The small size and unique biophysical properties of Nbs excel conventional antibody fragments for the recognition of uncommon or hidden epitopes and for binding into cavities or active sites of protein targets. Further, Nbs can be designed as bispecific and bivalent antibodies or attached to reporter molecules.⁽²⁸⁾ Nbs are stable, survive the gastro-intestinal system and can easily be manufactured. Therefore, Nbs can be used in many applications including drug discovery and therapy, but also as a versatile and valuable tool for purification, functional study and crystallization of proteins.⁽²⁹⁾

The nanobodies of the invention generally comprise a single amino acid chain that can be considered to comprise four “framework sequences” or FRs and three “complementary determining regions” or CDRs. The term “complementary determining region” or “CDR” refers to variable regions in nanobodies and contains the amino acid sequences capable of specifically binding to antigenic targets. These CDR regions account for the basic specificity of the nanobody for a particular antigenic determinant structure. Such regions are also referred to as “hypervariable regions.”

As used herein, the terms “complementarity determining region” or “CDR” refer to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains. The delineation of the CDR sequences is based on the IMGT unique numbering system for V-domains and V-like domains.⁽³⁵⁾

Non-limiting examples of such nanobodies according to the present invention are as described herein (see Table 4; SEQ ID NO:3, SEQ ID NO:4, SEQ ID NO:7, SEQ ID NO:8, SEQ ID NO:112, SEQ ID NO:114, SEQ ID NO:116). In a specific embodiment, the above nanobodies can comprise at least one of the complementary determining regions (CDRs). More specifically, the above nanobodies can be selected from the group comprising SEQ ID NO:3, SEQ ID NO:4, SEQ ID NO:7, SEQ ID NO:8, SEQ ID NO:112, SEQ ID NO:114, SEQ ID NO:116, or a functional fragment thereof. A functional fragment, as used herein, is one of the CDR loops. Preferably, the functional fragment is CDR3. More specifically, the nanobodies consist of any of SEQ ID NO:3, SEQ ID NO:4, SEQ ID NO:7, SEQ ID NO:8, SEQ ID NO:112, SEQ ID NO:114, SEQ ID NO:116. In still another embodiment, a nucleic acid sequence encoding any of the above nanobodies or functional fragments is also part of the present invention (for example, see Table 4; SEQ ID NO:1, SEQ ID NO:2, SEQ ID NO:5, SEQ ID NO:6, SEQ ID NO:111, SEQ ID NO:113, SEQ ID NO:115).

It should be noted that the term nanobody as used herein in its broadest sense is not limited to a specific biological source or to a specific method of preparation. For example, the nanobodies of the invention can generally be obtained: (1) by isolating the V_{H}H domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized V_{H}H domain; (4) by “camelization” of a naturally occurring VH domain from any animal species, and in particular from a mammalian species, such as from a human being, or by expression of a nucleic acid encoding such a camelized VH domain; (5) by “camelisation” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

One preferred class of nanobodies corresponds to the V_{H}H domains of naturally occurring heavy chain antibodies directed against a macrophage mannose receptor. As further described herein, such V_{H}H sequences can generally be generated or obtained by suitably immunizing a species of Camelid with a MMR, (i.e., so as to raise an immune response and/or heavy chain antibodies directed against a MMR), by obtaining a suitable biological sample from the Camelid (such as a blood sample, or any sample of B-cells), and by generating V_{H}H sequences directed against a MMR, starting from the sample, using any suitable technique known per se. Such techniques will be clear to the skilled person. Alternatively, such naturally occurring V_{H}H domains against MMR can be obtained from naive libraries of Camelid V_{H}H sequences, for example, by screening such a library using MMR or at least one part, fragment, antigenic determinant or epitope thereof using one or more screening techniques known per se. Such libraries and techniques are, for example, described in WO9937681, WO0190190, WO03025020 and WO03035694. Alternatively, improved synthetic or semi-synthetic libraries derived from naive V_{H}H libraries may be used, such as V_{H}H libraries obtained from naive V_{H}H libraries by techniques such as random mutagenesis and/or CDR shuffling, as for example, described in WO0043507. Yet another technique for obtaining V_{H}H sequences directed against a MMR involves suitably immunizing a transgenic mammal that is capable of expressing heavy chain antibodies (i.e., so as to raise an immune response and/or heavy chain antibodies directed against a MMR), obtaining a suitable biological sample from the transgenic mammal (such as a blood sample, or any sample of B-cells), and then generating V_{H}H sequences directed against a MMR starting from the sample, using any suitable technique known per se. For example, for this purpose, the heavy chain antibody-expressing mice and the further methods and techniques described in WO02085945 and in WO04049794 can be used.

A particularly preferred class of nanobodies of the invention comprises nanobodies with an amino acid sequence that corresponds to the amino acid sequence of a naturally occurring V_{H}H domain, but that has been “humanized,” i.e., by replacing one or more amino acid residues in the amino acid sequence of the naturally occurring V_{H}H sequence (and in particular in the framework sequences) by one or more of the amino acid residues that occur at the corresponding position(s) in a VH domain from a conventional 4-chain antibody from a human being. This can be performed in a manner known per se, which will be clear to the skilled person, for example, on the basis of the further description herein and the prior art on humanization referred to herein. Again, it should be noted that such humanized Nanobodies of the invention can be obtained in any suitable manner known per se (i.e., as indicated under points (1)-(8) above) and thus are not strictly limited to polypeptides that have been obtained using a polypeptide that comprises a naturally occurring V_{H}H domain as a starting material.

Another particularly preferred class of nanobodies of the invention comprises nanobodies with an amino acid sequence that corresponds to the amino acid sequence of a naturally occurring VH domain, but that has been “camelized,” i.e., by replacing one or more amino acid residues in the amino acid sequence of a naturally occurring VH domain from a conventional 4-chain antibody by one or more of the amino acid residues that occur at the corresponding position(s) in a V_{H}H domain of a heavy chain antibody. Such “camelizing” substitutions are preferably inserted at amino acid positions that form and/or are present at the VH-VL interface, and/or at the so-called Camelidae hallmark residues, as defined herein (see, for example, WO9404678). Preferably, the VH sequence that is used as a starting material or starting point for generating or designing the camelized nanobody is preferably a VH sequence from a mammal, more preferably the VH sequence of a human being, such as a VH3 sequence. However, it should be noted that such camelized nanobodies of the invention can be obtained in any suitable manner known per se (i.e., as indicated under points (1)-(8) above) and thus are not strictly limited to polypeptides that have been obtained using a polypeptide that comprises a naturally occurring VH domain as a starting material. For example, both “humanization” and “camelization” can be performed by providing a nucleotide sequence that encodes a naturally occurring V_{H}H domain or VH domain, respectively, and then changing, in a manner known per se, one or more codons in the nucleotide sequence in such a way that the new nucleotide sequence encodes a “humanized” or “camelized” nanobody of the invention, respectively. This nucleic acid can then be expressed in a manner known per se, so as to provide the desired nanobody of the invention. Alternatively, based on the amino acid sequence of a naturally occurring V_{H}H domain or VH domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring V_{H}H domain or VH domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired nanobody of the invention. Other suitable methods and techniques for obtaining the nanobodies of the invention and/or nucleic acids encoding the same, starting from naturally occurring VH sequences or preferably V_{H}H sequences, will be clear from the skilled person, and may, for example, comprise combining one or more parts of one or more naturally occurring VH sequences (such as one or more FR sequences and/or CDR sequences), one or more parts of one or more naturally occurring V_{H}H sequences (such as one or more FR sequences or CDR sequences), and/or one or more synthetic or semi-synthetic sequences, in a suitable manner, so as to provide a nanobody of the invention or a nucleotide sequence or nucleic acid encoding the same.

It is also within the scope of the invention to use natural or synthetic analogs, mutants, variants, alleles, homologs and orthologs (herein collectively referred to as “analogs”) of the nanobodies of the invention as defined herein, and in particular analogs of the nanobodies of SEQ ID NO:3, SEQ ID NO:4, SEQ ID NO:7, SEQ ID NO:8, SEQ ID NO:112, SEQ ID NO:114, SEQ ID NO:116 (see Table 4). Thus, according to one embodiment of the invention, the term “nanobody of the invention” in its broadest sense also covers such analogs. Generally, in such analogs, one or more amino acid residues may have been replaced, deleted and/or added, compared to the nanobodies of the invention as defined herein. Such substitutions, insertions or deletions may be made in one or more of the framework regions and/or in one or more of the CDRs, and in particular analogs of the CDRs of the nanobodies of SEQ ID NO:3, SEQ ID NO:4, SEQ ID NO:7, SEQ ID NO:8, SEQ ID NO:112, SEQ ID NO:114, SEQ ID NO:116.

By means of non-limiting examples, a substitution may, for example, be a conservative substitution (as described herein) and/or an amino acid residue may be replaced by another amino acid residue that naturally occurs at the same position in another V_{H}H domain. Thus, any one or more substitutions, deletions or insertions, or any combination thereof, that either improve the properties of the nanobody of the invention or that at least do not detract too much from the desired properties or from the balance or combination of desired properties of the nanobody of the invention (i.e., to the extent that the nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may, for example, involve introducing a limited number of possible substitutions and determining their influence on the properties of the nanobodies thus obtained.

For example, and depending on the host organism used to express the nanobody of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example, to allow site-specific pegylation.

One preferred class of analogs of the nanobodies of the invention comprise nanobodies that have been humanized (i.e., compared to the sequence of a naturally occurring nanobody of the invention). As mentioned in the background art cited herein, such humanization generally involves replacing one or more amino acid residues in the sequence of a naturally occurring V_{H}H with the amino acid residues that occur at the same position in a human VH domain, such as a human VH3 domain. Examples of possible humanizing substitutions or combinations of humanizing substitutions will be clear to the skilled person, from the possible humanizing substitutions mentioned in the background art cited herein, and/or from a comparison between the sequence of a nanobody and the sequence of a naturally occurring human VH domain. The humanizing substitutions should be chosen such that the resulting humanized nanobodies still retain the favourable properties of nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may, for example, involve introducing a limited number of possible humanizing substitutions and determining their influence on the properties of the nanobodies thus obtained. Generally, as a result of humanization, the nanobodies of the invention may become more “human-like,” while still retaining the favorable properties of the nanobodies of the invention as described herein. As a result, such humanized nanobodies may have several advantages, such as a reduced immunogenicity, compared to the corresponding naturally occurring V_{H}H domains. Again, based on the disclosure herein and optionally after a limited degree of routine experimentation, the skilled person will be able to select humanizing substitutions or suitable combinations of humanizing substitutions which optimize or achieve a desired or suitable balance between the favorable properties provided by the humanizing substitutions on the one hand and the favorable properties of naturally occurring V_{H}H domains on the other hand. Examples of such modifications, as well as examples of amino acid residues within the nanobody sequence that can be modified in such a manner (i.e., either on the protein backbone but preferably on a side chain), methods and techniques that can be used to introduce such modifications and the potential uses and advantages of such modifications will be clear to the skilled person. For example, such a modification may involve the introduction (e.g., by covalent linking or in another suitable manner) of one or more functional groups, residues or moieties into or onto the nanobody of the invention, and in particular of one or more functional groups, residues or moieties that confer one or more desired properties or functionalities to the Nanobody of the invention. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFvs and single domain antibodies), for which reference is, for example, made to Remington's Pharmaceutical Sciences, 16th ed., Mack Publishing Co., Easton, Pa. (1980). Such functional groups may, for example, be linked directly (for example, covalently) to a nanobody of the invention, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFvs); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965. Various reagents for pegylation of proteins are also commercially available, for example, from Nektar Therapeutics, USA. Preferably, site-directed pegylation is used, in particular via a cysteine-residue (see, for example, Yang et al., Protein Engineering, 16, 10, 761-770 (2003). For example, for this purpose, PEG may be attached to a cysteine residue that naturally occurs in a nanobody of the invention, a nanobody of the invention may be modified so as to suitably introduce one or more cysteine residues for attachment of PEG, or an amino acid sequence comprising one or more cysteine residues for attachment of PEG may be fused to the N- and/or C-terminus of a nanobody of the invention, all using techniques of protein engineering known per se to the skilled person. Preferably, for the nanobodies and proteins of the invention, a PEG is used with a molecular weight of more than 5000, such as more than 10,000 and less than 200,000, such as less than 100,000; for example, in the range of 20,000-80,000. Another, usually less preferred modification comprises N-linked or O-linked glycosylation, usually as part of co-translational and/or post-translational modification, depending on the host cell used for expressing the nanobody or polypeptide of the invention. Another technique for increasing the half-life of a nanobody may comprise the engineering into bifunctional nanobodies (for example, one nanobody against the target MMR and one against a serum protein such as albumin) or into fusions of nanobodies with peptides (for example, a peptide against a serum protein such as albumin).

Yet another modification may comprise the introduction of one or more detectable labels or other signal-generating groups or moieties, depending on the intended use of the labeled nanobody. Suitable labels and techniques for attaching, using and detecting them will be clear to the skilled person, and for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta- V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person, and for example, include moieties that can be detected using NMR or ESR spectroscopy. Such labeled nanobodies and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the nanobody of the invention to another protein, polypeptide or chemical compound that is bound to the other half of the binding pair, i.e., through formation of the binding pair. For example, a nanobody of the invention may be conjugated to biotin, and linked to another protein, polypeptide, compound or carrier conjugated to avidin or streptavidin. For example, such a conjugated nanobody may be used as a reporter, for example, in a diagnostic system where a detectable signal-producing agent is conjugated to avidin or streptavidin. Such binding pairs may, for example, also be used to bind the nanobody of the invention to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the nanobody of the invention.

The nanobodies of the present invention may generally be directed against any MMR, and may in particular be directed against the ectodomain of any MMR. It is further expected that the nanobodies according to this aspect of the invention will generally bind to all naturally occurring or synthetic analogs, variants, mutants, alleles of the MMR.

In a particular embodiment, the nanobody of the invention is bivalent and formed by bonding, chemically or by recombinant DNA techniques, together two monovalent single domain of heavy chains. In another particular embodiment the nanobody of the invention is bi-specific and formed by bonding together two variable domains of heavy chains, each with a different specificity. Similarly, polypeptides comprising multivalent or multi-specific nanobodies are included here as non-limiting examples. Preferably, a monovalent nanobody of the invention is such that it will bind to the MMR (as described herein) with an affinity less than 500 nM, preferably less than 200 nM, more preferably less than 10 nM, such as less than 500 pM. Also, according to this aspect, any multivalent or multispecific (as defined herein) nanobody of the invention may also be suitably directed against two or more different epitopes on the same antigen, for example, against two different parts of the ectodomain. Such multivalent or multispecific nanobodies of the invention may also have (or be engineered and/or selected for) increased avidity and/or improved selectivity for the desired MMR, and/or for any other desired property or combination of desired properties that may be obtained by the use of such multivalent or multispecific nanobodies.

As used herein, the term “affinity” refers to the degree to which an immunoglobulin, such as an antibody, or an immunoglobulin fragment, such as a nanobody binds to an antigen so as to shift the equilibrium of antigen and antibody (fragment) toward the presence of a complex formed by their binding. Thus, where an antigen and antibody (fragment) are combined in relatively equal concentration, an antibody (fragment) of high affinity will bind to the available antigen so as to shift the equilibrium toward high concentration of the resulting complex.

Further, the invention also relates to a pharmaceutical composition comprising a therapeutically effective amount of a nanobody of the invention, and at least one of pharmaceutically acceptable carrier, adjuvant or diluent.

A “carrier,” or “adjuvant,” in particular, a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. So, pharmaceutically acceptable carriers are inherently non-toxic and nontherapeutic, and they are known to the person skilled in the art. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Carriers or adjuvants may be, as a non limiting example, Ringer's solution, dextrose solution or Hank's solution. Non aqueous solutions such as fixed oils and ethyl oleate may also be used. A preferred excipient is 5% dextrose in saline. The excipient may contain minor amounts of additives such as substances that enhance isotonicity and chemical stability, including buffers and preservatives.

As used herein, the terms “therapeutically effective amount,” “therapeutically effective dose” and “effective amount” mean the amount needed to achieve the desired result or results.

As used herein, “pharmaceutically acceptable” means a material that is not biologically or otherwise undesirable, i.e., the material may be administered to an individual along with the compound without causing any undesirable biological effects or interacting in a deleterious manner with any of the other components of the pharmaceutical composition in which it is contained.

Certain of the above-described nanobodies may have therapeutic utility and may be administered to a subject having a condition in order to treat the subject for the condition.

Accordingly, in a second aspect, the invention relates to a method of preventing and/or treating cancer, comprising administrating a pharmaceutically effective amount of a nanobody of the invention or a pharmaceutical composition derived thereof to a mammal in need thereof.

As used herein, the term “preventing cancer” means inhibiting or reversing the onset of the disease, inhibiting or reversing the initial signs of the disease, inhibiting the appearance of clinical symptoms of the disease. As used herein, “treating cancer” or “treating a subject or individual having cancer” includes substantially inhibiting the disease, substantially slowing or reversing the progression of the disease, substantially ameliorating clinical symptoms of the disease or substantially preventing the appearance of clinical symptoms of the disease. In particular, it includes inhibition of the replication of cancer cells, inhibition of the spread of cancer, reduction in tumor size, lessening or reducing the number of cancerous cells in the body, and/or amelioration or alleviation of the symptoms of cancer. A treatment is considered therapeutic if there is a decrease in mortality and/or morbidity, and may be performed prophylactically, or therapeutically. A variety of subjects or individuals are treatable. Generally such individuals are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the individuals will be humans.

As used herein, the term “cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, and gastrointestinal or stomach cancer.

In a more specific aspect, the invention relates to a method of inhibiting tumor growth or tumor metastases in a mammal in need thereof comprising selectively targeting TAM subpopulations linked to different intratumoral regions, such as hypoxic or normoxic regions of a solid tumor. As a specific embodiment, the above method comprises administering to the mammal a pharmaceutically effective amount of a nanobody or a pharmaceutical composition according to the invention, in particular a nanobody fused to a toxin, or to a cytotoxic drug, or to an enzyme capable of converting a prodrug into a cytotoxic drug, or to a radionuclide, or coupled to a cytotoxic cell, and the like (see also Example section).

According to particular embodiments, a TAM subpopulation can be defined as MHC II^{low} or MHC II^{hi}. In a preferred embodiment, the TAM subpopulation is defined as MHC II^{low}. For a detailed description of different TAM subpopulations, reference is made to the Example section, in particular Examples 1 to 8.

The nanobody and/or pharmaceutical composition may be administered by any suitable method within the knowledge of the skilled man. The administration of a nanobody as described above or a pharmaceutically acceptable salt thereof may be by way of oral, inhaled or parenteral administration. In particular embodiments the nanobody is delivered through intrathecal or intracerebroventricular administration. The active compound may be administered alone or preferably formulated as a pharmaceutical composition. An amount effective to treat a certain disease or disorder that express the antigen recognized by the nanobody depends on the usual factors such as the nature and severity of the disorder being treated and the weight of the mammal. However, a unit dose will normally be in the range of 0.01 to 50 mg, for example, 0.01 to 10 mg, or 0.05 to 2 mg of nanobody or a pharmaceutically acceptable salt thereof. Unit doses will normally be administered once or more than once a day, for example, 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example, 0.01 to 10 mg or more usually 0.05 to 10 mg. It is greatly preferred that the compound or a pharmaceutically acceptable salt thereof is administered in the form of a unit-dose composition, such as a unit dose oral, parenteral, or inhaled composition. Such compositions are prepared by admixture and are suitably adapted for oral, inhaled or parenteral administration, and as such may be in the form of tablets, capsules, oral liquid preparations, powders, granules, lozenges, reconstitutable powders, injectable and infusable solutions or suspensions or suppositories or aerosols. Tablets and capsules for oral administration are usually presented in a unit dose, and contain conventional excipients such as binding agents, fillers, diluents, tableting agents, lubricants, disintegrants, colorants, flavorings, and wetting agents. The tablets may be coated according to well known methods in the art. Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate. These solid oral compositions may be prepared by conventional methods of blending, filling, tableting or the like. Repeated blending operations may be used to distribute the active agent throughout those compositions employing large quantities of fillers. Such operations are, of course, conventional in the art. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example, sorbitol, syrup, methyl cellulose, gelatin, hydroxyethylcellulose, carboxymethyl cellulose, aluminum stearate gel or hydrogenated edible fats, emulsifying agents, for example, lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example, methyl or propyl p-hydroxybenzoate or sorbic acid, and if desired conventional flavoring or coloring agents. Oral formulations also include conventional sustained release formulations, such as tablets or granules having an enteric coating. Preferably, compositions for inhalation are presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, for example, between 1 and 5 microns, such as between 2 and 5 microns. A favored inhaled dose will be in the range of 0.05 to 2 mg, for example, 0.05 to 0.5 mg, 0.1 to 1 mg or 0.5 to 2 mg. For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilizing before filling into a suitable vial or ampoule and sealing. Advantageously, adjuvants such as a local anesthetic, preservatives and buffering agents are also dissolved in the vehicle. To enhance the stability, the composition can be frozen after filling into the vial and the water removed under vacuum. Parenteral suspensions are prepared in substantially the same manner except that the compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound. Where appropriate, small amounts of bronchodilators, for example, sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. As is common practice, the compositions will usually be accompanied by written or printed directions for use in the medical treatment concerned. All these medicaments can be intended for human or veterinary use.

The efficacy of the nanobodies of the invention, and of compositions comprising the same, can be tested using any suitable in vitro assay, cell-based assay, in vivo assay and/or animal model known per se, or any combination thereof, depending on the specific disease or disorder involved.

In a specific embodiment it should be clear that the therapeutic method of the present invention against cancer can also be used in combination with any other cancer therapy known in the art such as irradiation, chemotherapy or surgery.

Reliable hypoxia tracers that can be used for non-invasive tumor imaging are currently unavailable or limiting. The availability of such tracers would represent a significant progress in the field of radiotherapy, since they would allow the radiotherapist to adapt the radiation dose, depending on the targeted tumor region (hypoxic versus normoxic). The identification of tumor-associated macrophage (TAM) subsets that are situated in hypoxic/normoxic environments allows for the identification of macrophage-specific biomarkers that can be used for non-invasive imaging of hypoxic/normoxic areas in tumors. For example, MMR represents such a marker, since it is preferentially expressed on the hypoxic MHC II^{low} TAMs. Due to their small size and high tumor penetrance, nanobodies are the ideal format for non-invasive imaging. Nanobodies raised against markers that are preferentially expressed on the hypoxic MHC II^{low} TAMs can be used for the imaging of hypoxia in tumors. The anti-MMR nanobodies can be used in this respect.

Other applications of TAM subset-specific nanobodies, coupled to tracers for imaging (for example, Near Infrared Fluorescent or NIRF tracers), include but are not limited to (i) accurately quantifying the amount of TAM or TAM subsets inside any given tumor, which can be of prognostic value, (ii) assessing the impact of therapy—including TAM-directed therapies as presently claimed—on the amount and/or the activation state of TAM, (iii) visualizing hypoxic/normoxic regions within the tumor.

Accordingly, in still another aspect, the invention also relates to a method of imaging tumor cells in a mammal suffering from or suspected to suffer from cancer comprising selectively visualizing TAM subpopulations linked to hypoxic or normoxic regions in a solid tumor. As a specific embodiment, the method comprises administering to the mammal a nanobody fused to a detectable label.

Further, in still another aspect, the invention relates to a method of diagnosing or prognosing cancer aggressiveness in a subject or individual suffering from or suspected to suffer from cancer comprising determining the relative percentage of TAM subpopulations in a sample from the subject or individual. In particular, the method comprises the steps of (i) providing a sample from the individual comprising cancer cells or suspected to comprise cancer cells; (ii) determining in the sample the relative percentage of TAM subpopulations; (iii) classifying the individual as having a good/prognosis or diagnosing the individual as having cancer according to the results of step (ii). To further illustrate this, reference is made to Example 19.

A sample may comprise any clinically relevant tissue sample, such as a tumor biopsy or fine needle aspirate, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines. The sample may also be paraffin-embedded tissue sections. It is understood that the cancer tissue includes the primary tumor tissue as well as a organ-specific or tissue-specific metastasis tissue.

In the context of the present invention, prognosing an individual suffering from or suspected to suffer from cancer refers to a prediction of the survival probability of individual having cancer or relapse risk which is related to the invasive or metastatic behavior (i.e., malignant progression) of tumor tissue or cells. As used herein, “good prognosis” means a desired outcome. For example, in the context of cancer, a good prognosis may be an expectation of no recurrences or metastasis within two, three, four, five years or more of initial diagnosis of cancer. “Poor prognosis” means an undesired outcome. For example, in the context of cancer, a poor prognosis may be an expectation of a recurrence or metastasis within two, three, four, or five years of initial diagnosis of cancer. Poor prognosis of cancer may indicate that a tumor is relatively aggressive, while good prognosis may indicate that a tumor is relatively nonaggressive.

As used herein, the terms “determining,” “measuring,” “assessing,” and “assaying” are used interchangeably and include both quantitative and qualitative determinations.

The following examples more fully illustrate preferred features of the invention, but are not intended to limit the invention in any way. Those having ordinary skill in the art and access to the teachings herein will recognize additional modifications and embodiments within the scope thereof. Therefore, the present invention is limited only by the claims attached herein. All of the starting materials and reagents disclosed below are known to those skilled in the art, and are available commercially or can be prepared using well-known techniques.

### EXAMPLES

### Material and Methods to the Examples

### Mice and Cell Lines

Female Balb/c mice were purchased from Harlan. Balb/c CX₃CR1^{GFP/GFP} mice were a gift from Dr. Grégoire Lauvau (Université de Nice-Sophia Antipolis, France) and Dr. Frédéric Geissmann (King's College London, UK). All animal studies were approved by and performed according to the guidelines of the institutional review board. The Balb/c mammary adenocarcinoma cell line TS/A⁽¹⁰⁾ was provided by Dr. Vincenzo Bronte (Istituto Oncologico Veneto, Italy) and was injected subcutaneously (sc) in the flank (3×10⁶ cells).

### Tumor Preparation, Flow Cytometry and Cell Sorting

Tumors were chopped and incubated for 25 minutes (37° C.) with 10 U/ml Collagenase type1, 400 U/ml Collagenase typeIV and 30 U/ml DNAsel (Worthington). Density gradients (Axis-Shield) were used to remove tissue debris and dead cells.

Commercial antibodies used for cell surface stainings are found in Table 2. Non-labeled anti-CCR2 (MC-21) was a gift of Dr. Matthias Mack (University of Regensburg, Germany). To prevent a specific binding, rat anti-mouse CD16/CD32 (clone 2.4G2, BD Biosciences) was used.

To purify TAMs, CD11b⁺ cells were isolated via MACS using anti-CD11b microbeads (Miltenyi Biotec). Subsequently, cells were sorted using a BD FACSAria™ II (BD Biosciences).

### In Vivo Labeling of Blood Monocytes

Latex labeling of blood monocytes was performed as described earlier.^{(19,20)} Briefly, to label Ly6C^{low} monocytes and track their infiltration in tumors, mice were injected intravenously (iv) with 250 μl of 0.5 μm fluoresbrite yellow-green microspheres (Polysciences) diluted 1:25 in PBS. 24 hours later, mice received sc TS/A injections. To label and track Ly6C^{hi} monocytes, mice were injected iv with 250 μl of clodronate liposomes. 18 hours later, mice received iv latex injection and sc TS/A injection. Clodronate was a gift from Roche and was incorporated into liposomes as previously described.⁽²¹⁾

### Bromodeoxyuridine Labeling and Ki67 Stainings

Tumor-bearing mice (14 days pi) were given an initial intraperitoneal injection of 1 mg BrdU (BD Biosciences), followed by continuous BrdU administration in the drinking water at a concentration of 0.8 mg/ml (Sigma). Tumors were collected after consecutive time points and BrdU intracellular stainings were performed following the manufacturer's instructions (BrdU labeling Kit, BD Biosciences). PE-labeled anti-Ki67 or matching isotype controls (BD Biosciences) was added together with FITC-labeled anti-BrdU in the final step of the intracellular staining protocol.

RNA Extraction, cDNA Preparation and Quantitative Real-Time PCR

RNA was extracted using TRIzol (Invitrogen) and was reverse-transcribed with oligo(dT) and SuperScript II RT (Invitrogen), following the manufacturer's instructions. Quantitative real-time PCR was performed in an iCycler, with iQ SYBR Green Supermix (Bio-Rad) using gene-specific primers (Table 2). PCR cycles consisted of 1-minute denaturation at 94° C., 45-second annealing at 55° C., and 1-minute extension at 72° C. Gene expression was normalized according to the expression of ribosomal protein S12.

Intracellular TNFα and iNOS Stainings

For intracellular TNFα stainings, freshly isolated TAMs were cultured in vitro for 1 hour, after which Brefeldin A (BD Biosciences) was added. 5 hours later cells were fixed, permeabilized (Fix/Perm kit, eBioScience) and stained with anti-TNFα. For intracellular iNOS stainings, freshly isolated TAMs were cultured in vitro with or without 10 U/ml IFNγ and/or 10 ng/ml LPS. 12 hours later cells were fixed, permeabilized and stained with anti-iNOS. Normalized delta-Median Fluorescence Intensity (ΔMFI) was calculated as follows: [(MFI iNOS staining)−(MFI isotype staining)]/(MFI iNOS staining). FACS data were acquired using a BD FACSCanto II (BD Biosciences).

### Measurement of Arginase Activity

The arginase activity in the lysate of 5 10⁵ sorted TAMs was measured as described earlier.⁽²²⁾

### Immunohistochemistry and Hypoxia Measurements

For hypoxia stainings, tumor-bearing mice were injected with 80 mg/kg body weight pimonidazole (Hypoxyprobe-1, HP-1, HPI Inc.) and 2 hours later tumors were collected.

For immunohistochemistry, tumors were snap-frozen in liquid nitrogen and 5 μm sections were made. Sections were fixed for 10 minutes in ice-cold aceton. To block aspecific binding sites, sections were incubated 30 minutes with 10% normal donkey serum (Jackson ImmunoResearch Laboratories). For CD11b, MHC II and anti-HP-1 triple stainings, sections were: (1) incubated 30 minutes with purified rat anti-CD11b (BD Biosciences) and purified rabbit anti-HP-1 (HPI Inc.) (2) incubated 30 minutes with F(ab′)₂ donkey anti-Rat/Cy3 (Jackson ImmunoResearch Laboratories) and F(ab′)₂ donkey anti-rabbit/Cy5 (Jackson ImmunoResearch Laboratories) (3) remaining anti-rat binding sites were blocked with 5% normal rat serum (Jackson ImmunoResearch Laboratories) (4) incubated 30 minutes with rat anti-MHC II/alexa-fluor 488 (M5/114.15.2 Biolegend). Rat anti-MECA32 (Pan-endothelial cell antigen) was from BD Biosciences. Sections were mounted with fluorescent mounting medium (Dako). Pictures were acquired with a Plan-Neofluar 10×/0.30 or Plan-Neofluar 20×/0.50 (Carl Zeiss) objective on a Zeiss Axioplan 2 microscope (Carl Zeiss) equipped with an Orca-R2 camera (Hamamatsu) and Smartcapture 3 software (Digital Scientific UK). For flow cytometric HP-1 measurements, tumor single cell suspensions were made, and cells were fixed and permeabilized using the BD Biosciences Fix/Perm kit. Finally, rat anti-HP1/FITC(HPI Inc.) was added for 30 minutes at 37° C.

### Determining Latex Phagocytosis in Vivo and in Vitro

For measuring in vivo latex uptake by TAMs, tumor-bearing mice were injected iv with 250 μl of yellow-green latex microspheres (Polysciences) diluted 1:25 in PBS. 1-2 hours later, tumor single cell suspensions were made and latex uptake by tumor CD11b⁺ cells was assessed via FACS. For in vitro latex uptake, freshly isolated TAMs were cultured in 96-well plates for 40 minutes at 4° C. or 37° C., in the presence of latex (diluted 1:5000).

### Chorioallantoic Membrane Angiogenesis Assays

Chorioallantoic membrane (CAM) assays were performed as described earlier.⁽²³⁾ Briefly, fertilized white leghorn chicken eggs (Wyverkens, Halle, Belgium) were incubated at 37° C. for 3 days prior to removing 3 ml of albumen to detach the shell from the developing CAM. Next, a window was made in the eggshell to expose the CAM. At day 9, sterile absorbable gelatin sponges (1-2 mm³; Hospithera, Brussels, Belgium) were impregnated with 5×10⁴ sorted TAM subsets and placed on the CAM. Sponges were also loaded with PBS/0.1% BSA (1 mg/ml, ˜50 μg/embryo) as negative control and with recombinant human VEGF-A₁₆₅ (100 μg/ml, ˜5 μg/embryo) as positive control. At day 13, membranes were fixed with 4% paraformaldehyde and the area around the implants was analyzed using a Zeiss Lumar V.12 stereomicroscope with NeoLumar S 1.5× objective (15× magnification). Digital images were captured using an AxioCam MRc5 and processed with Axiovision 4.5 Software (Zeiss). To determine the number of blood vessels, a grid containing three concentric circles with diameters of 4, 5, and 6 mm was positioned on the surface of the CAM and all vessels radiating from the sample spot and intersecting the circles were counted under a stereomicroscope.

### DQ-OVA Processing, MLR Assays, Suppression Assays

To assess TAM antigen processing, tumor single cell suspensions were incubated for 15 minutes at 0° C. or 37° C. in the presence of 10 μg/ml DQ-OVA (Molecular Probes), allowing for antigen uptake. After thorough washing, cells could further process DQ-OVA intracellularly during different time intervals, at 0° C. or 37° C. Following each time interval, cells were surface labeled and DQ-OVA fluorescence in each TAM subset was measured via FACS.

For Mixed Leukocyte Reaction (MLR) assays, T cells were purified from C57BL/6 spleens, by first depleting CD11c⁺ and CD19⁺ cells on a MACS LD column using anti-CD11c and anti-CD19 microbeads (Miltenyi biotech) and subsequently positively selecting CD4⁺ or CD8⁺ T cells using anti-CD4 or anti-CD8 microbeads (Miltenyi biotech). 2×10⁵ purified C57BL/6 T cells were cultured with 5×10⁴ sorted Balb/c TAMs or cDCs, in round-bottom 96-well plates. 3 days later ³H-thymidine was added and cells were allowed to proliferate for another 18 hours before incorporated radioactivity was measured.

For T-cell suppression assays, 1×10⁵ (1:2), 5×10⁴ (1:4), 2.5×10⁴ (1:8) or 1.25×10⁴ (1:16) sorted TAMs or cDCs were added to 2×10⁵ naive Balb/c splenocytes, in flat-bottom 96-well plates. These cocultures were promptly stimulated with 1 μg/ml anti-CD3, 24 hours later ³H-thymidine was added and cells were allowed to proliferate for another 18 hours before incorporated radioactivity was measured. L-NMMA (0.5 mM, Sigma), nor-NOHA (0.5 mM, Calbiochem), or both, were added from the beginning of the culture. The Relative % suppression of proliferation was calculated as described earlier⁽²⁴⁾: (% Suppression without inhibitor)/(% Suppression with inhibitor)×100, with % Suppression calculated as [1−(proliferation of splenocytes)/(proliferation splenocytes+TAMs)]×100.

### Sorting of Splenic Conventional DCs

To purify splenic conventional DCs, spleens were flushed with 200 U/ml collagenase III (Worthington) and squashed. Subsequently, CD11c⁺ cells were enriched via MACS, using anti-CD11c microbeads (Miltenyi Biotec), after which CD11c⁺MHC II^{hi}B220⁻Ly6C⁻ DCs were sorted using a BD FACSAria™ II (BD Biosciences).

### Statistics

Statistical significance was determined by the Student's t test, using Microsoft Excel or GraphPad Prism 4.0 software. Differences were considered significant when P≦0.05. Geometric means and confidence intervals were determined using Microsoft Excel.

### Generation of Mono- and Bivalent Anti-MMR Nanobodies

The anti-MMR Nanobody (Nb) clone 1 was isolated from an immune phage library in a similar way as described before.^{(30,31)} In brief, an alpaca (Vicugna pacos) was immunized with 100 μg MMR (R&D Systems) six times at weekly intervals. mRNA prepared from peripheral blood lymphocytes was used to make cDNA with the Ready-to-G0 You-prime-first-strand beads (GE Healthcare). The gene sequences encoding the VHHs were PCR amplified using the CALL001/CALL002 and A6E/38 primer pairs. These PCR fragments were ligated into the pHEN4 phagemid vector after digestion with the PstI and BstEII restriction enzymes. Using M13K07 helper phage infection, the VHH library was expressed on phages and specific Nanobody-phages were enriched by several consecutive rounds of in vitro selection on microtiter plates (Nunc). Individual colonies were screened in ELISA for antigen recognition with non-specific phage particles serving as a negative control. The VHH genes of the clones that scored positive in ELISA were recloned into the expression vector pHEN6 using the restriction enzymes PstI and BstEII. Expression in the periplasm and purification of Nanobody was performed as described previously.⁽²⁸⁾

Bivalent Nanobodies were generated by recombinantly attaching a linker sequence 3′ of the VHH sequence using PCR primer biNbF (5′-CCG GCC ATG GCC CAG GTG CAG CTT CAG GAG TCT GG AGG AGG-3′; SEQ ID NO:117) and primers biNbG4SR (5′-TGA TTC CTG CAG CTG CAC CTG ACT ACC GCC GCC TCC AGA TCC ACC TCC GCC ACT ACC GCC TCC GCC TGA GGA GAC GGT GAC CTG GGT C-3′; SEQ ID NO:118), biNbg2cR (5′-TGA TTC CTG CAG CTG CAC CTG TGC CAT TGG AGC TTT GGG AGC TTT GGA GCT GGG GTC TTC GCT GTG GTG CGC TGA GGA GAC GGT GAC CTG GGT C-3′; SEQ ID NO:119), biNbigAR (5′-TGA TTC CTG CAG CTG CAC CTG ACT TGC CGG TGG TGT GGA TGG TGA TGG TGT GGG AGG TGT AGA TGG GCT TGA GGA GAC GGT GAC CTG GGT C-3′; SEQ ID NO:120) which code for a (G₄S)₃ (GGGGSGGGGSGGGGS; SEQ ID NO:121), llama IgG2 hinge (AHHSEDPSSKAPKAPMA; SEQ ID NO:122) or human IgA hinge (SPSTPPTPSPSTPPAS; SEQ ID NO:123) linker respectively. These PCR fragments were inserted 5′ of the VHH gene in the original VHH expression vector with a PstI/BstEII restriction digest. After ligation, the resulting bivalent anti-MMR Nanobody vector was expressed as described above.

### Construction and Production Anti-MMR-PE38 Immunotoxins

Anti-MMR-PE38 toxin fusions were generated using the anti-MMR bivalent Nanobodies as templates. The PE38 (recombinant Pseudomonas Exotoxin A⁽³³⁾ gene was PCR amplified from the pET28aCD11scFv-PE38 vector⁽³²⁾ using the PE38HF (5′-ATT GAA TTC TAT TAG TGG TGG TGG TGG TGG TGC TCG AGT G-3; SEQ ID NO:124) and PE38bisR (5′-TTA ACT GCA GAT GGC CGA AGA GGG CGG CAG CCT-3′; SEQ ID NO:125) primers. During this PCR reaction a PstI and EcoRI restriction site were introduced 5′ and 3′ of the PE38 gene respectively. Both the PE38 PCR fragments and the pHEN6 vectors containing bivalent anti-MMR Nanobody genes with a (G₄S)₃ (GGGGSGGGGSGGGGS; SEQ ID NO:121), IIama IgG2 hinge (AHHSEDPSSKAPKAPMA; SEQ ID NO:122) or human IgA hinge (SPSTPPTPSPSTPPAS; SEQ ID NO:123) linker were digested using PstI and EcoRI restriction enzymes. By ligating the PE38 gene fragment in the pHEN6 vector fragments, the PE38 gene was fused to the 3′ end of the anti-MRR Nanobody-linker gene. The resulting immunotoxin constructs were produced and purified in the same manner as the mono- and bivalent anti-MMR Nanobody constructs.

### Surface Plasmon Resonance

Affinity analysis was performed using a BIAcore T100 (GE Healthcare) with HEPES-buffered saline running buffer (10 mM HEPES with 0.15 M NaCl, 3.4 mM EDTA and 0.005% surfactant P20 at pH 7.4). MRR was immobilized on a CM5 chip in acetate buffer 50 mM (pH 5,0), resulting in 2100 RU MMR coated on the chip. A second channel on the same chip was activated/deactivated in a similar way and served as a negative control. The MMR Nanobodies were used as analytes in 11 different concentrations, ranging from 1 to 2000 nM, at a flow rate of 10 ml/min. Glycine-HCl 50 mM (pH 2.0) was used for elution. The kinetic and equilibrium parameters (kd, ka and K_{D}) values were calculated from the combined sensogram of all concentrations using BIAcore T100 evaluation software 2.02 (GE Healthcare).

### Nanobody Purification

All Nanobody proteins were purified from E. coli periplasmic extracts using immobilized metal affinity chromatography (IMAC) on Ni-NTA resin (Sigma-Aldrich, St. Louis, Mo.) followed by size exclusion chromatography (SEC) on Superdex 75 HR 10/30 (Pharmacia, Gaithersburg, Md.) in phosphate buffered saline pH 7.4 (PBS).

Nanobody Labeling and in Vitro Characterization of ^{99m}Tc-Labeled Nanobodies

Nanobodies were labeled with ^{99m}Tc at their hexahistidine tail. For the labeling, [^{99m}Tc(H₂O)₃(CO)₃]⁺ was synthesized by adding 1 mL of ^{99m}TcO4⁻ (0.74-3.7 GBq) to an Isolink kit (Mallinckrodt Medical BV) containing 4.5 mg of sodium boranocarbonate, 2.85 mg of sodium tetraborate. 10H₂O, 8.5 mg of sodium tartrate. 2H₂O, and 7.15 mg of sodium carbonate, pH 10.5. The vial was incubated at 100° C. in a boiling bath for 20 min. The freshly prepared [^{99m}Tc(H₂O)₃(CO)₃]⁺ was allowed to cool at room temperature for 5 min and neutralized with 125 μL of 1 M HCl to pH 7-8. [^{99m}Tc(H₂O)₃(CO)₃]⁺ was added to 50 μL of 1 mg/mL monovalent Nanobody or 2 mg/ml bivalent Nanobody, together with 50 μL of carbonate buffer, pH 8. The mixture was incubated for 90 min at 52° C. in a water bath. The labeling efficiency was determined by instant thin-layer chromatography in acetone as mobile phase and analyzed using a radiometric chromatogram scanner (VCS-201; Veenstra). When the labeling yield was less than 90%, the ^{99m}Tc-Nanobody solution was purified on a NAP-5 column (GE Healthcare) pre-equilibrated with phosphate-buffered saline (PBS) and passed through a 0.22 μm Millipore filter to eliminate possible aggregates.

### Pinhole SPECT-MicroCT Imaging Procedure

Mice were intravenously injected with 100-200 μl 45-155 MBq (about 5-10 μg) of ^{99m}Tc-Nanobody, with or without an excess of concentrated monovalent or bivalent unlabled Nanobody. Mice were anesthetized with a mixture of 18.75 mg/kg ketamine hydrochloride (Ketamine 1000®, CEVA, Brussels, Belgium) and 0.5 mg/kg medetomidin hydrochloride (Domitor®, Pfizer, Brussels, Belgium) 10-15 min before pinhole SPECT acquisition.

MicroCT imaging was followed by pinhole SPECT on separate imaging systems. MicroCT was performed using a dual source CT scanner (Skyscan 1178, Skyscan, Aartselaar, Belgium) with 60 kV and 615 mA at a resolution of 83 μm. The total body scan time was 2 minutes. Image reconstruction was performed using filtered backprojection (Nrecon, Skyscan, Aartselaar, Belgium). Total body pinhole SPECT was performed at 60 min or 180 min post-injection (p.i.) using a dual headed gamma camera (e.cam¹⁸⁰ Siemens Medical Solutions, Ill., USA), mounted with two multi-pinhole collimators (3 pinholes of 1.5 mm in each collimator, 200 mm focal length, 80 mm radius of rotation). Images were acquired over 360 degrees in 64 projections of 10 s into 128×128 matrices resulting in a total imaging time of 14 min. The SPECT images were reconstructed using an iterative reconstruction algorithm (OSEM) modified for the three pinhole geometry and automatically reoriented for fusion with CT based on six ⁵⁷Co landmarks.

### Image Analysis

Image viewing and quantification was performed using AMIDE Medical Image Data Examiner software. Ellipsoid regions of interest (ROIs) were drawn around the tumor and major organs. Uptake was calculated as the counts in the tissue divided by the injected activity counts and normalized for the ROI size (% IA/cm³).

### Biodistribution Analysis

30 min after microCT/SPECT acquisition, mice were sacrificed with a lethal dose of pentobarbital (Nembutal; CEVA). Tumor, kidneys, liver, lungs, muscle, spleen, lymph nodes, bone, heart, and blood were removed and weighed, and the radioactivity was measured using an automated λ-counter (Cobra II Inspector 5003; Canberra-Packard). Tissue and organ uptake was calculated as percentage of injected activity per gram of tissue (% IA/g), corrected for decay.

### Example 1

### TS/A Tumors are Highly Infiltrated with a Heterogeneous Population of Myeloid Cells Containing Distinct Granulocyte and Monocyte/Macrophage Subsets

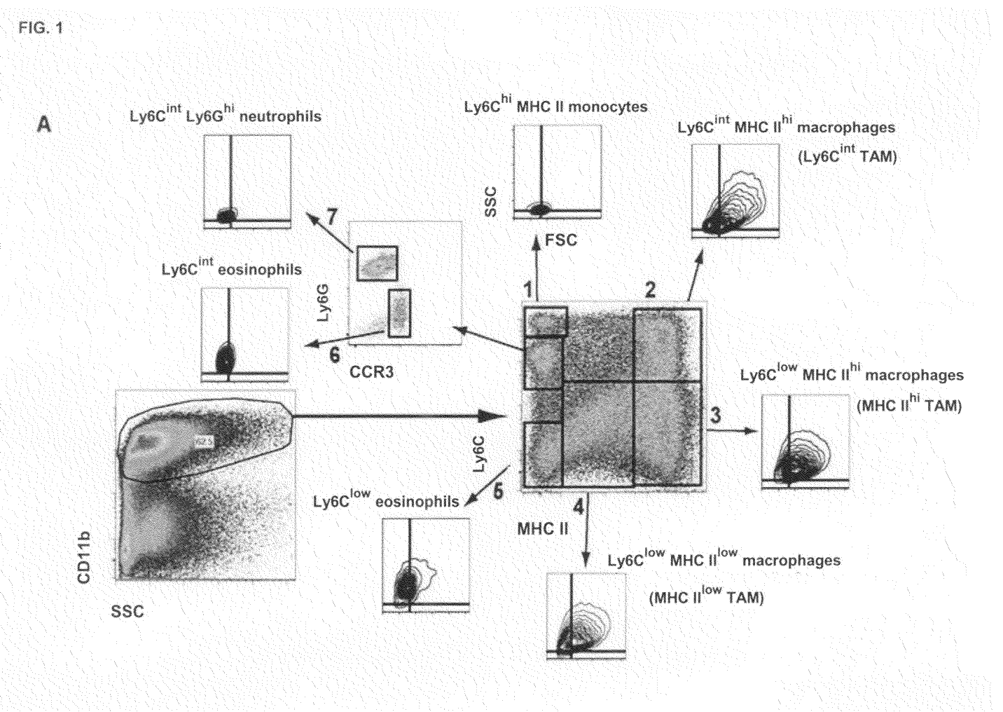

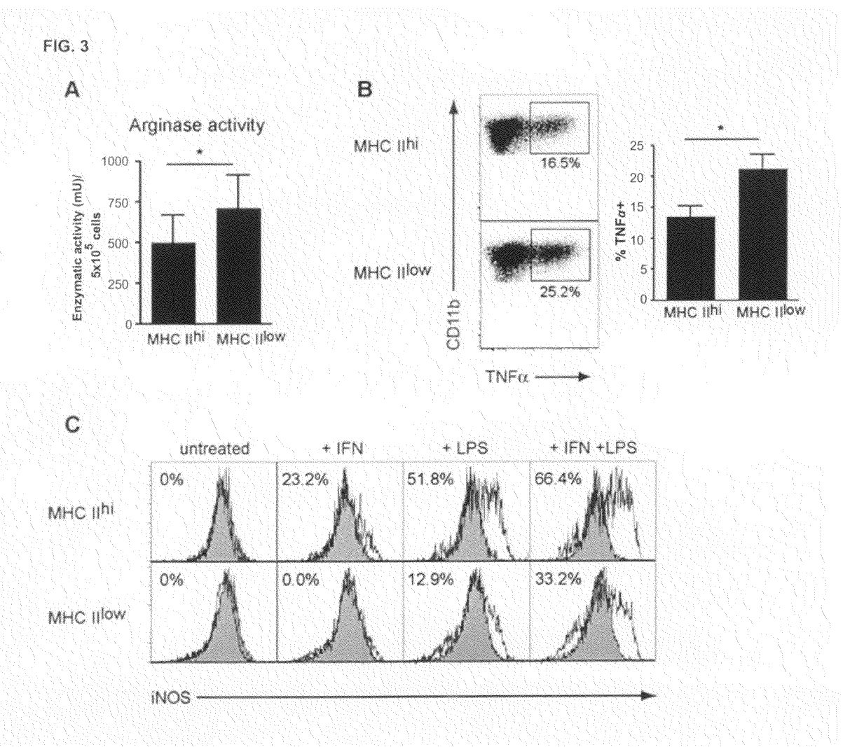

To study the tumor-infiltrating myeloid compartment, we employed the Balb/c mammary adenocarcinoma model TS/A. Subcutaneous tumors contained a large CD11b⁺ fraction, indicating a high infiltration of myeloid cells (FIG. 1A). Interestingly, this CD11b⁺ population was heterogeneous and encompassed at least 7 subsets, which could be readily distinguished based on their differential expression of MHC class II and Ly6C (FIG. 1A). Ly6C^{hi}MHC II⁻ cells (Gate 1: FIG. 1A) were F4/80⁺CX₃CR1^{low}CCR2^{hi}CD62L⁺, did not express the granulocyte markers Ly6G or CCR³ and had a small size and granularity (FSC^{low}SSC^{low}), indicating that they were Ly6C^{hi} monocytes (FIG. 1A,C and FIG. 6). The CD11b⁺MHC II⁺ cells in Gates 2-4 were reminiscent of macrophages, having an enlarged macrophage-like scatter and expressing high levels of F4/80 (FIG. 1 A,C). Remarkably, distinct subsets of tumor-associated macrophages (TAMs) were clearly distinguishable: Ly6C^{int}MHC II^{hi} (Ly6C^{int} TAMs, Gate 2), Ly6C^{low}MHC II^{hi} (MHC II^{hi} TAMs, Gate 3) and Ly6C^{low}MHC II^{low} (MHC II^{low} TAMs, Gate 4). The majority of Ly6C^{low}MHC II⁻ cells were CCR3⁺CX₃CR1⁻ eosinophils (Gate 5: FIG. 1A and Gate E: FIG. 6). However, Ly6C^{low}MHC II⁻ cells also consisted of CCR3⁻CX₃CR1^{low} (Gate 2: FIG. 6) and CCR3⁻CX₃CR1^{hi} (Gate 3: FIG. 6) cells, the latter possibly resembling Ly6C^{low}CX₃CR1^{hi} monocytes. However, the majority of these CX₃CR1^{hi} cells did not have a monocyte scatter, suggesting they were TAMs (FIG. 6). This suggests that Ly6C^{low} monocytes were not present in significant amounts in these tumors. Finally, TS/A tumors were also infiltrated with CCR3⁺Ly6C^{int} eosinophils (Gate 6: FIG. 1A), and Ly6G^{hi} neutrophils (Gate 7: FIG. 1A).

Interestingly, the relative percentages of these distinct myeloid subpopulations dramatically changed as tumors progressed (FIG. 1B). Within the TAM compartment, the percentage of Ly6C^{int} TAMs decreased, while the Ly6C^{low}MHC II^{low} TAM subset became gradually more prominent, reaching up to 60% of the myeloid tumor-infiltrate in large tumors (>10 mm).

### Example 2

### Ly6C^{hi} Monocytes are the Precursors of all TAM Subsets in TS/A Tumors

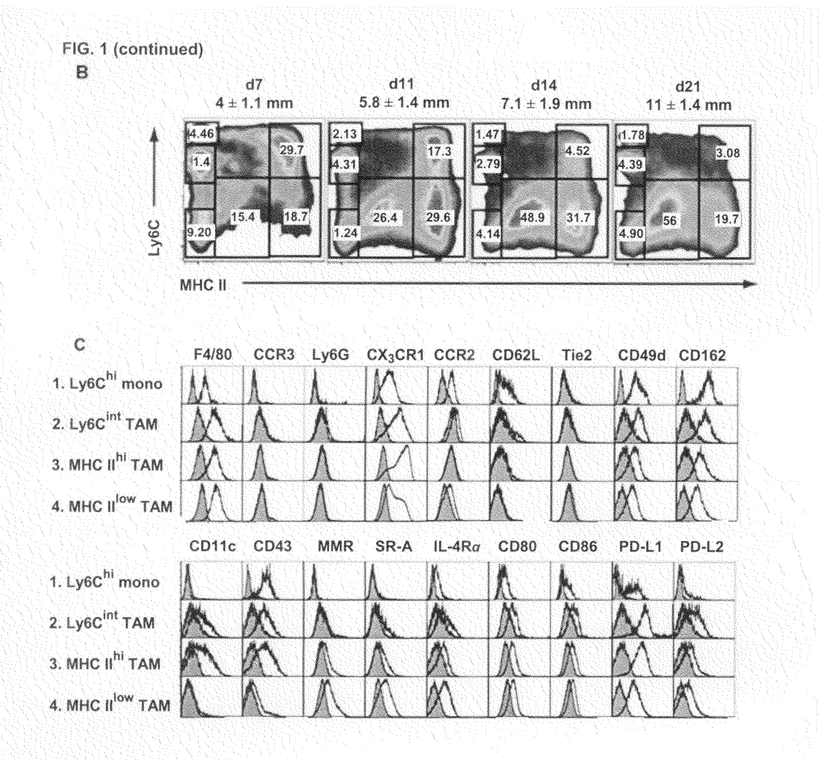

Macrophages typically derive from circulating blood-borne precursors such as monocytes. The presence of Ly6C^{hi}, but not Ly6C^{low}, monocytes in TS/A tumors suggested that the former could be more efficiently recruited to tumors and function as the TAM precursor. To investigate this, we selectively labeled Ly6C^{hi} or Ly6C^{low} monocyte subsets in vivo with fluorescent latex beads, using a previously described procedure.^{(11,12)} This method has been validated to stably label the respective monocyte subsets for 5-6 days in naïve mice. Hence, TS/A was injected after Ly6C^{low} or Ly6C^{hi} monocyte labeling and tumors were collected 6 days pi. No appreciable numbers of tumor-infiltrating latex⁺ monocytes were observed when applying the Ly6C^{low} labeling strategy (FIG. 2A). In contrast, Ly6C^{hi} labeling resulted in the detection of a significant fraction of CD11b⁺latex⁺ monocytes, illustrating that Ly6C^{hi} monocytes are the main tumor-infiltrating monocyte subset. With this approach, latex⁺ cells could be detected up to 19 days post tumor injection (FIG. 2B), allowing a follow-up of the monocyte progeny in the course of tumor growth. At day 6, latex⁺Ly6C^{hi} monocytes had differentiated into latex⁺Ly6C^{int} TAMs, and to some extent also into latex⁺MHC II^{hi} and latex⁺MHC II^{low} TAMs (FIG. 2B). From day 12 onward, the majority of latex⁺Ly6C^{hi} monocytes had converted into latex⁺MHC II^{hi} and latex⁺MHC II^{low} TAMs. Together, these data demonstrate that all TAM subsets can be derived from Ly6C^{hi} monocytes.

### Example 3

### Ly6C^{int}, MHC II^{hi} and MHC II^{low} TAMs have Distinct Differentiation Kinetics and Turnover Rates

To determine the turnover rate and differentiation kinetics of the monocyte/TAM subsets, BrdU was administered continuously to tumor-bearing animals and its incorporation was measured at consecutive time points. Tumor-infiltrating Ly6C^{hi} monocytes quickly became BrdU⁺, reaching plateau values after 48 hours of BrdU administration (FIG. 2D). This indicates a rapid monocyte turnover rate and/or proliferation of monocytes inside tumors. Remarkably, intratumoral Ly6C^{hi} monocytes were Ki67⁺, suggesting a proliferative potential (FIG. 2C). In contrast, TAMs were non-proliferating (Ki67⁻) and hence unable to directly incorporate BrdU. Therefore, BrdU⁺TAMs must differentiate from BrdU⁺monocytes, resulting in a lag phase of BrdU positively. Indeed, only a minor fraction of MHC II^{hi} and MHC II^{low} TAMs were BrdU⁺ upon 24 hours BrdU administration (FIG. 2D). However, compared with these subsets, Ly6C^{int} TAMs incorporated BrdU at a faster rate, with a higher percentage being BrdU⁺ already at 24 hours. These results suggest that monocytes first give rise to Ly6C^{int} TAMs, which then differentiate into MHC II^{hi} and MHC II^{low} TAMs. MHC II^{hi} and MHC II^{low} TAMs incorporated BrdU slowly and with similar kinetics, arguing for a comparable and low turnover rate.

### Example 4

### MHC II^{hi} and MHC II^{low} TAMs Differ at the Molecular Level

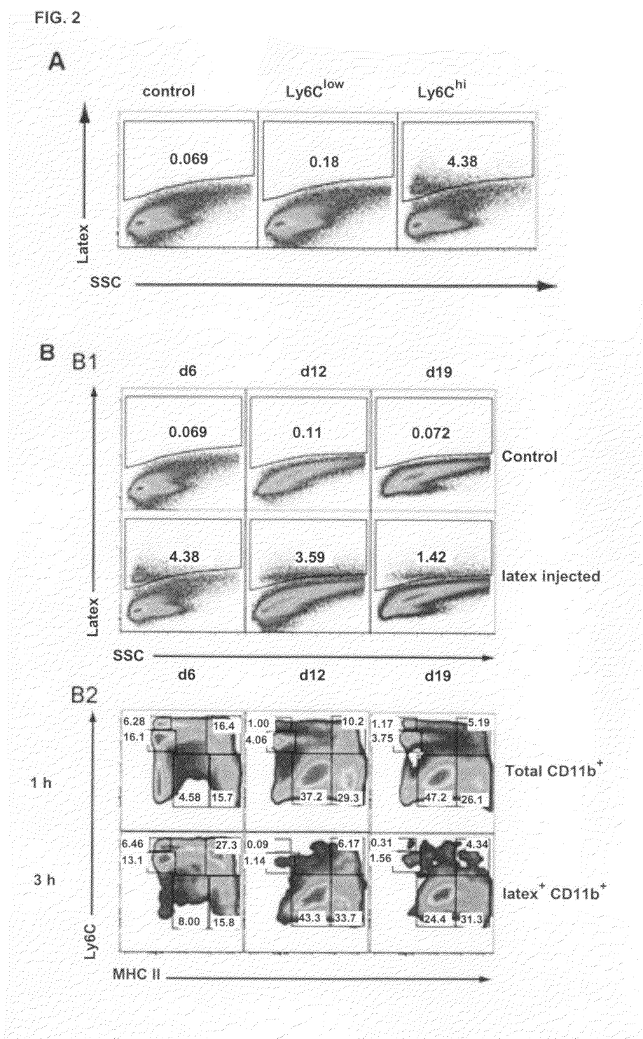

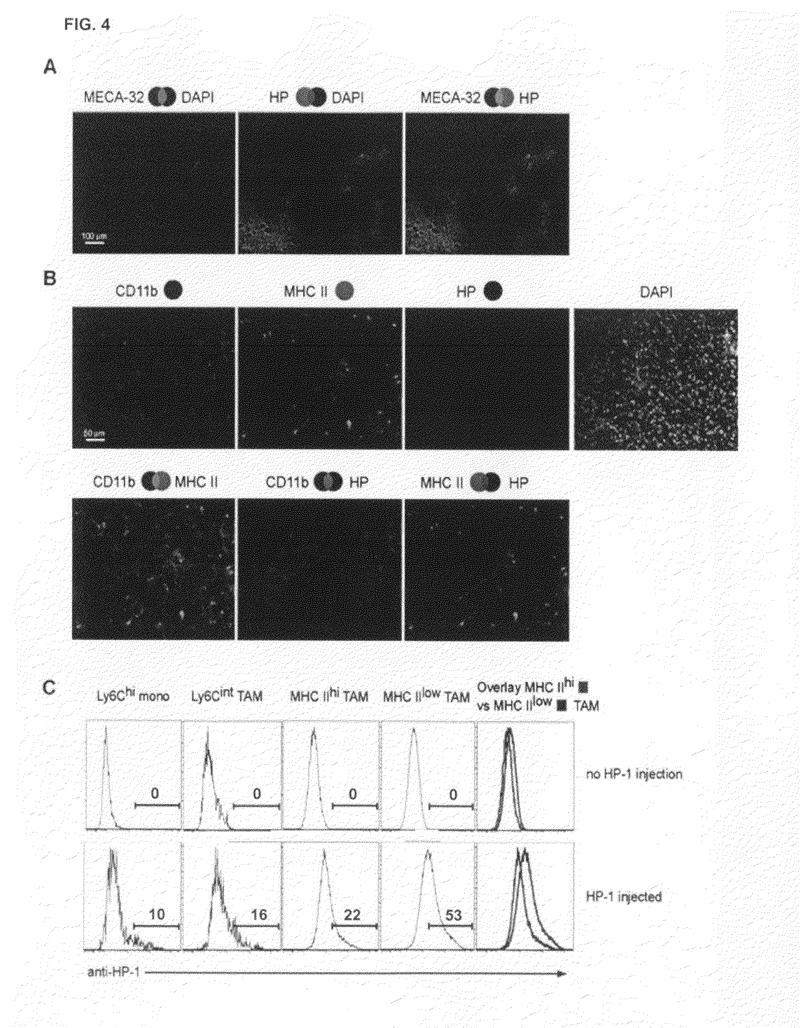

Efforts have been made before to characterize TAMs at the molecular level.^{(13,14)} We characterized the distinct TAM subsets at the gene and protein level. The gene expression of sorted MHC and MHC II^{low} TAMs (FIG. 7A) was analyzed via qRT-PCR (Table 1). Ly6C^{int} TAMs, constituting only a minor fraction in larger tumors, were not included in this analysis. Interestingly, when comparing MHC II^{hi} with MHC II^{low} TAMs (Table 1 hi/low), M2-associated genes such as Arg1 (Arginase-1), Cd163, Stab1 (Stabilin-1) and Mrc1 (MMR) were higher expressed in the MHC II^{low} subset. In contrast, more M1-type, pro-inflammatory genes such as Nos2 (iNOS), Ptgs2 (Cox2), II1b, II6 and II12b were upregulated in MHC II^{hi} TAMs. This differential activation state was also reflected at the protein level. Membrane expression of the M2 markers macrophage mannose receptor (MMR), macrophage scavenger receptor 1 (SR-A) and IL-4Rα were clearly higher on MHC II^{low} TAMs, while the M1-associated marker CD11c, was only expressed on MHC II^{hi} TAMs (FIG. 1C). Moreover, while arginase activity was observed in both TAM subsets, it was significantly higher for MHC II^{low} TAMs (FIG. 3A). In the same vein, TNFα which has previously been reported to associate with a M2 phenotype in tumors,^{(15,16)} was produced by both TAM subsets, but a significantly higher percentage of MHC II^{low} TAMs were found to be TNFα⁺ (FIG. 3B). While iNOS protein was not detected in freshly isolated TAMs, it could be induced by IFN-γ and/or LPS stimulation (FIG. 3C). Interestingly, IFN-γ or LPS induced iNOS more efficiently in MHC II^{hi} TAMs, with a higher fraction of these cells becoming iNOS⁺. Together, these data indicate that the identified TAM subsets have a differential activation state, with MHC II^{low} TAMs being more M2-oriented.

TAM subsets also showed a markedly distinct chemokine expression pattern (Table 1). Notably, mRNAs for chemokines typically involved in lymphocyte attraction, such as Cc15, Cx₃c11, Cxc111, Cxc110, Cxc19 and the CCR4 ligands Cc117 and Cc122 were upregulated in MHC II^{hi} TAMs. In contrast, mRNAs for monocyte/macrophage chemoattractants, such as Cc16, the CCR2 ligands Cc17, Cc12 and Cc112 and the CCR5/CCR1 ligands Cc14, Cc13 and Cc19 were significantly higher in MHC II^{low} TAMs. Furthermore, at the protein level, a differential expression of the chemokine receptors CX₃CR1 and CCR2 was observed, with MHC II^{hi} TAMs being CX₃CR1^{hi}CCR2⁻, while MHC II^{low} TAMs were CX₃CR1^{low}CCR2⁺ (FIG. 1C).

Both TAM subsets expressed many potentially pro-angiogenic genes, including Vegfa, Mmp9, Pgf; Spp1 and cathD (Table 1). However, several angiostatic factors such as angpt2, Cxc19, Cxc110 and Cxc111 were upregulated in the MHC II^{hi} fraction. One of the most differentially expressed genes (higher in MHC II^{low} TAMs) was Lyve1.

We conclude that MHC II^{hi} and MHC II^{low} TAMs have a distinguishing profile of molecules involved in inflammation (M1/M2), chemotaxis and angiogenesis.

### Example 5

### MHC II^{low} TAMs are Enriched in Regions of Hypoxia, While MHC II^{low} TAMs are Mainly Normoxic

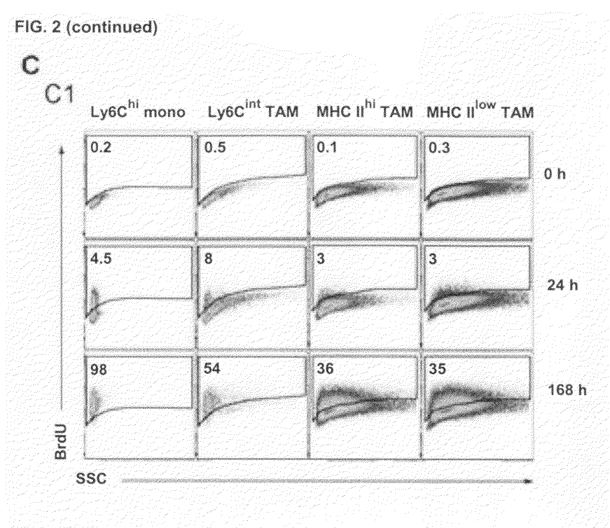

Tumors often harbor regions of hypoxia, a factor which is known to influence macrophage function.⁽⁹⁾ To visualize hypoxia in TS/A tumors, tumor-bearing mice were injected with pimonidazole (Hypoxyprobe-1, HP-1) and tumor sections were stained for hypoxic adducts and blood vessels. FIG. 4A shows that tumors indeed contained a large number of hypoxic cells, primarily in regions with a less developed vasculature. Interestingly, staining sections for HP-1, CD11b and MHC II demonstrated that many CD11b⁺MHC II⁻ cells (which in large tumors are mainly MHC II^{low} TAMs) were HP-1⁺ (FIG. 4B). Interestingly however, the majority of CD11b⁺MHC II⁺ cells were HP-1⁻. This indicates that while a significant fraction of MHC II^{low} TAMs resided in hypoxic areas, MHC II^{hi} TAMs were mainly normoxic. Importantly, HP-1 adducts could also be detected through intracellular flow cytometry on freshly isolated TAMs. Again, the highest signal was seen in MHC II^{low} TAMs, confirming they were the most hypoxic TAM subset (FIG. 4C).

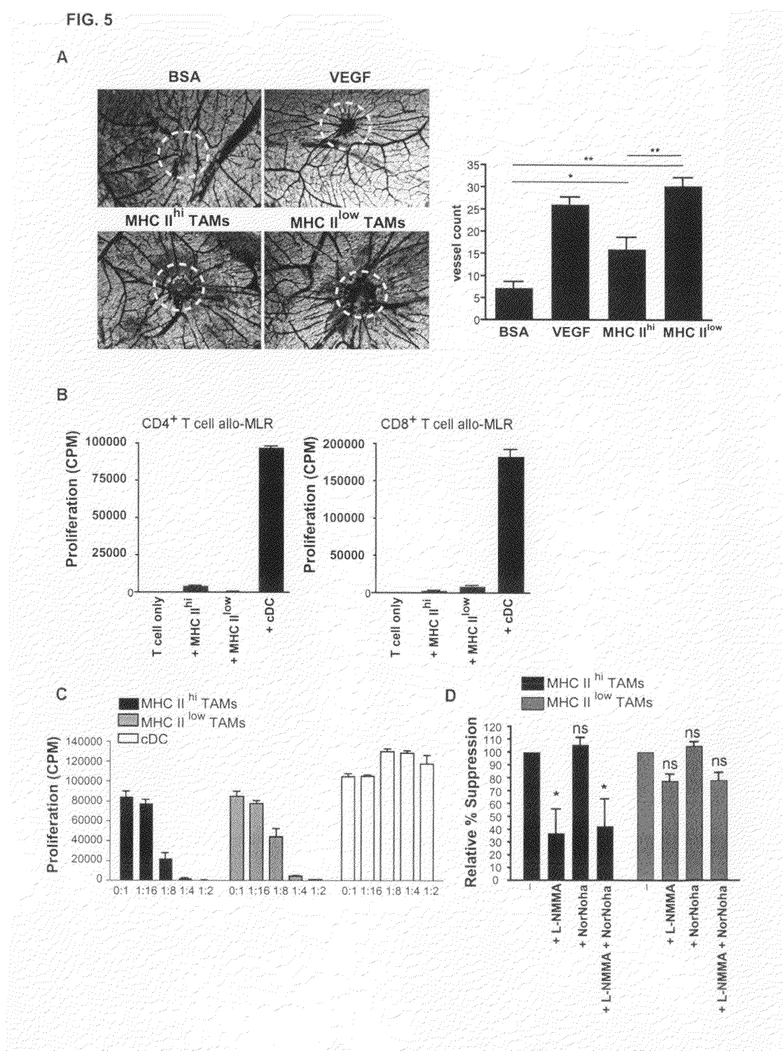

A consequence of MHC II^{low} TAMs being in hypoxic regions should be a reduced access to blood-transported molecules. To test this, fluorescent latex particles were injected iv in tumor-bearing mice. 1 to 2 hours later a fraction of tumor-associated CD11b⁺ cells were found to be latex⁺ (FIG. 8A). However, latex uptake was not equal in all TAM subsets. Indeed, in relative terms, MHC II^{low} TAMs phagocytosed less latex than monocytes and other TAM subsets. This was not due to an inherently reduced phagocytic capacity of MHC II^{low} TAMs, since the latter showed the highest phagocytic latex uptake in vitro (FIG. 8B). These data suggest that the reduced in vivo latex uptake of MHC II^{low} TAMs was due to a restricted access to latex particles which further substantiates the enrichment of MHC II^{low} TAMs in hypoxic regions.

### Example 6

### MHC II^{low} TAMs Show a Superior Pro-Angiogenic Activity in Vivo

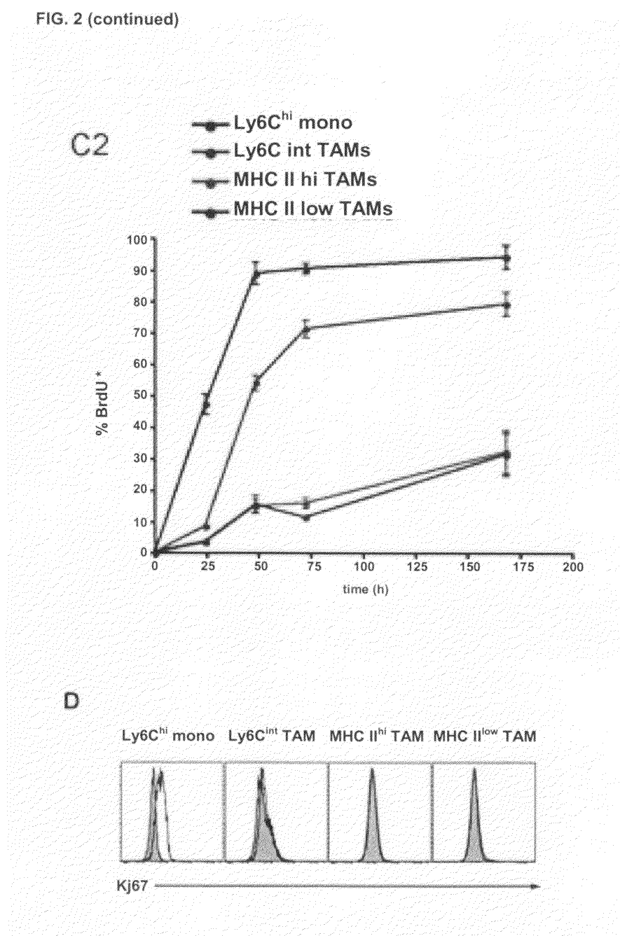

Hypoxia initiates an angiogenic program.⁽¹⁷⁾ In addition, our gene profiling revealed the expression of angiogenesis-regulating molecules in TAMs. To directly test the pro-angiogenic activity of both TAM subsets in vivo, we employed the chorioallantoic membrane (CAM) assay. Sorted MHC II^{hi} or MHC II^{low} TAMs were implanted on developing CAMs, while BSA or rhVEGF served as negative and positive controls, respectively. rhVEGF induced the outgrowth of allantoic vessels specifically directed towards the implants (FIG. 5A). Interestingly, compared with BSA controls, the presence of MHC II^{hi} or MHC II^{low} TAMs significantly increased the number of implant-directed vessels, demonstrating a pro-angiogenic activity for both TAM subsets. However, the vessel count for implants containing MHC II^{low} TAMs was on average 2-fold higher than with MHC II^{hi} TAMs. These data show that MHC II^{low} TAMs had a superior pro-angiogenic activity in vivo.

### Example 7

### TAMs are Poor Antigen-Presenters, but can Efficiently Suppress T-Cell Proliferation

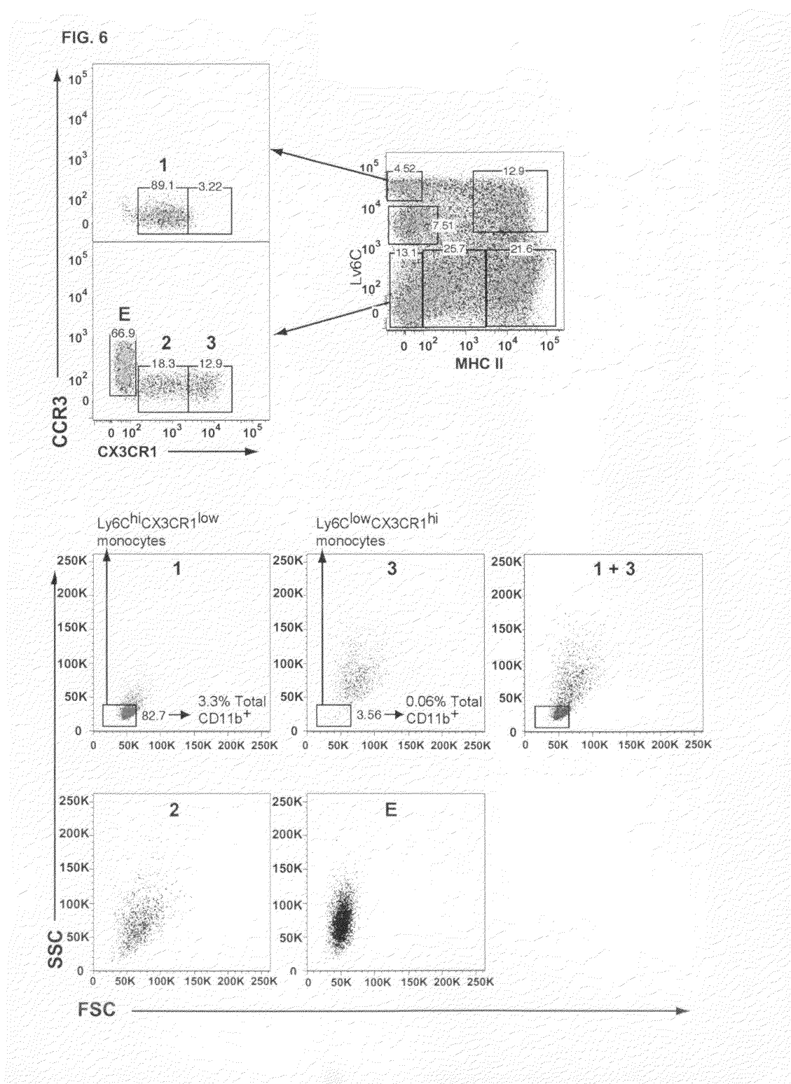

We wondered whether the TAM subsets were able to process internalized antigens and activate T cells. Both TAM subsets took up and processed DQ-Ovalbumin (DQ-OVA) at 37° C. However, examining DQ-OVA processing at consecutive time points indicated that processing naïve more slowly in the MHC II^{low} fraction (FIG. 9). To investigate whether TAMs could directly activate naïve T cells, a mixed leukocyte reaction (MLR) assay was used. Hereto, sorted MHC II^{hi} or MHC II^{low} TAMs were cultured with purified allogeneic C57BL/6 CD4⁺ or CD8⁺ T cells. Sorted splenic CD11c^{hi}MHC II^{hi} conventional DCs (cDCs) (FIG. 7B) were used as a reference T-cell-stimulating population.⁽¹⁸⁾ Compared with cDCs, MHC II^{hi} or MHC II^{low} TAMs induced poor proliferation of allogeneic CD4⁺ or CD8⁺ T cells (FIG. 5B), suggesting a limited antigen-presenting capacity or, alternatively, a T-cell suppressive capacity that overrules antigen-presentation.

To investigate the latter possibility, T cells were polyclonally activated in the presence of TAMs or cDCs. Interestingly, as opposed to cDCs, both MHC II^{hi} and MHC II^{low} TAMs equally suppressed anti-CD3-induced T-cell proliferation in a dose-dependent manner (FIG. 5C). In an attempt to identify the suppressive molecules responsible for TAM-mediated suppression, inhibitors of iNOS (L-NMMA) and arginase (N or Noha) were added to the co-cultures (FIG. 5D). Blocking iNOS significantly reduced T-cell suppression by MHC II^{hi} TAMs, demonstrating a role for nitric oxide in its suppressive mechanism. In contrast, iNOS inhibition only had a minor effect on the suppressive potential of MHC II^{low} TAMs, showing that both subsets employ different T-cell suppressive mechanisms.

### Example 8

### Similar TAM Subsets in Other Tumor Models

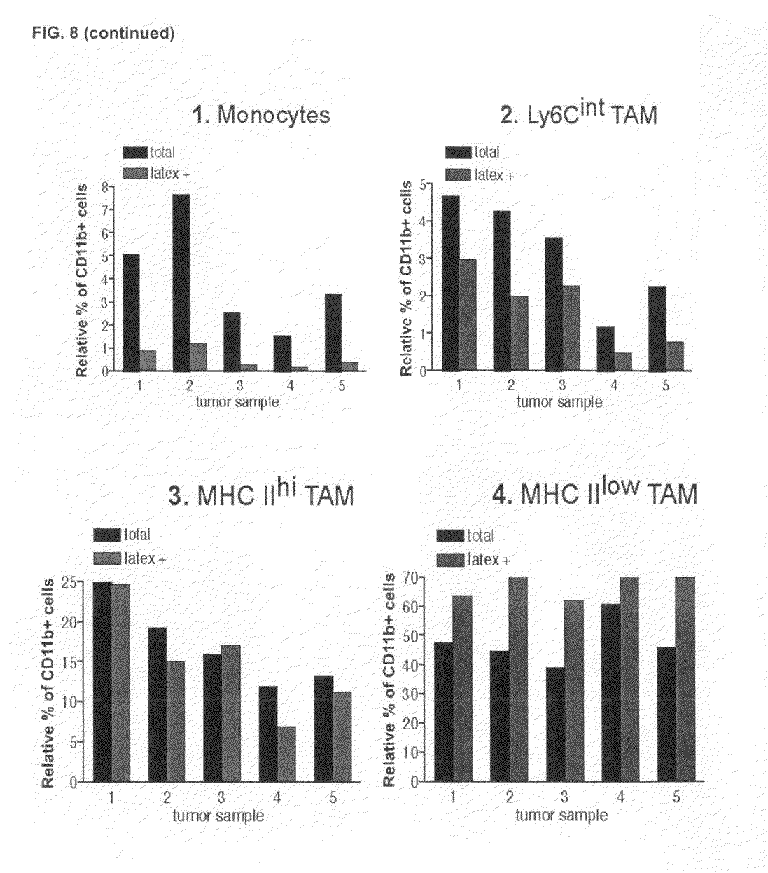

Interestingly, the TAM subsets identified in TS/A tumors, were also present in other tumor models. Both in the Lewis Lung Carcinoma (LLC) model and in the mammary carcinoma model 4T1, MHC II^{hi} and MHC II^{low} TAMs could be identified (FIG. 13A). Furthermore, as in TS/A, typical M2 markers such as MMR and IL4Rα were higher expressed on MHC II^{low} TAMs, while M1 markers such as CD11c were higher on MHC II^{hi} TAMs (FIG. 13B). This indicates that our initial findings in TS/A are not restricted to a single tumor model or even to a single carcinoma type (mammary vs. lung carcinoma). The dynamics of TAM subsets in the LLC model resembled that of TS/A, with MHC II^{low} TAMs accumulating over time and forming the majority of myeloid cells in established tumors (FIG. 13C, LLC). However, 4T1 tumors did not adhere to this trend and instead MHC II^{hi} TAMs accumulated as tumors progressed (FIG. 13C, 4T1). These data indicate that the accumulation of TAM subsets over time can vary from one tumor type to another, which possibly reflects differences in tumor architecture. Therefore, these findings provide a rationale for classifying tumors based on the relative percentage of TAM subsets (with tumor volume taken into account). This might be useful for devising a tailored therapy and/or as a prognostic factor.

### Example 9

### Nanobodies Against the Macrophage Mannose Receptor (CD206-MMR)

As outlined in the Examples above, TAMs can adopt different phenotypes and functional specializations. For example, TAMs located in hypoxic tumor regions were found to be extremely pro-angiogenic, suggesting that they play an important role in tumor vascularization. Interestingly, we have identified CD206 (macrophage mannose receptor) as a membrane marker which is specifically expressed on this tumor-promoting TAM subset. Anti-CD206 (anti-MMR) nanobodies, which are the smallest available antigen-binding entities, were created (see also Example 14) in order to target these cells in vivo. It was shown that the newly created anti-CD206 Nbs bind strongly to TAMs, but not to other myeloid cell types such as monocytes and granulocytes or any other tumor resident cells. These and other nanobodies against any of the markers of Table 1 are used for non-invasive imaging of TAMs using SPECT/Micro-CT. These nanobodies are also used to create immunotoxins for the therapeutical targeting of these cells in pre-clinical tumor models or for antibody-directed enzyme prodrug therapies (ADEPT).

### Example 10

### In Vivo Imaging Using Macrophage Mannose Receptor Nanobodies

In a next step, we performed in vivo imaging using Macrophage Mannose Receptor (MMR) targeting nanobodies. The nanobodies were labeled at their hexahistidine-tail with ^{99m}Tc at elevated temperatures by tricarbonyl-chemistry. Purified, ^{99m}Tc-labeled Nanobodies were injected intravenously in mice and total body scans were made using pinhole SPECT and microCT.

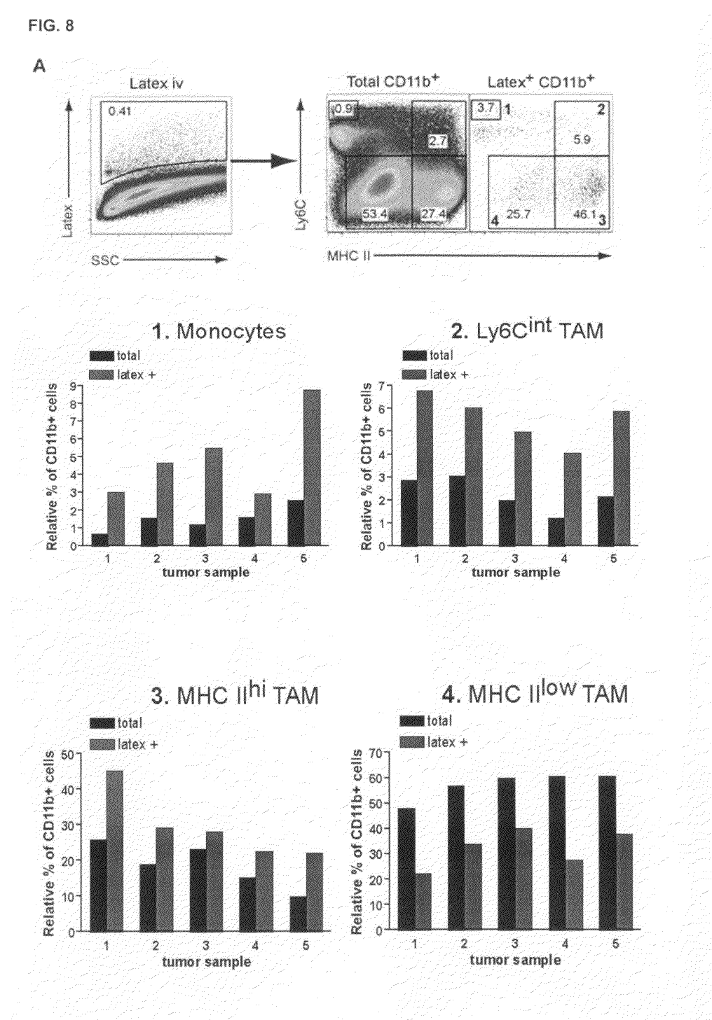

The first step in the in vivo evaluation was the study of the biodistribution in healthy mice. This allows to evaluate physiological sites of specific accumulation and to determine the pharmacokinetic properties of the imaging probes. MMR nanobodies show uptake in organs such as lungs, spleen and liver. The blood clearance is fast with less than 1% IA (injected activity)/ml remaining in blood at 1 hour 30 minutes post injection. We also tested MMR nanobodies in MMR knock-out mice where the uptake in liver and spleen dropped below 1% 1A/g (FIG. 11). These data indicate that the accumulation in organs such as liver and spleen is related to MMR expression and therefore specific. Only the accumulation in lungs appears to be MMR-unrelated.

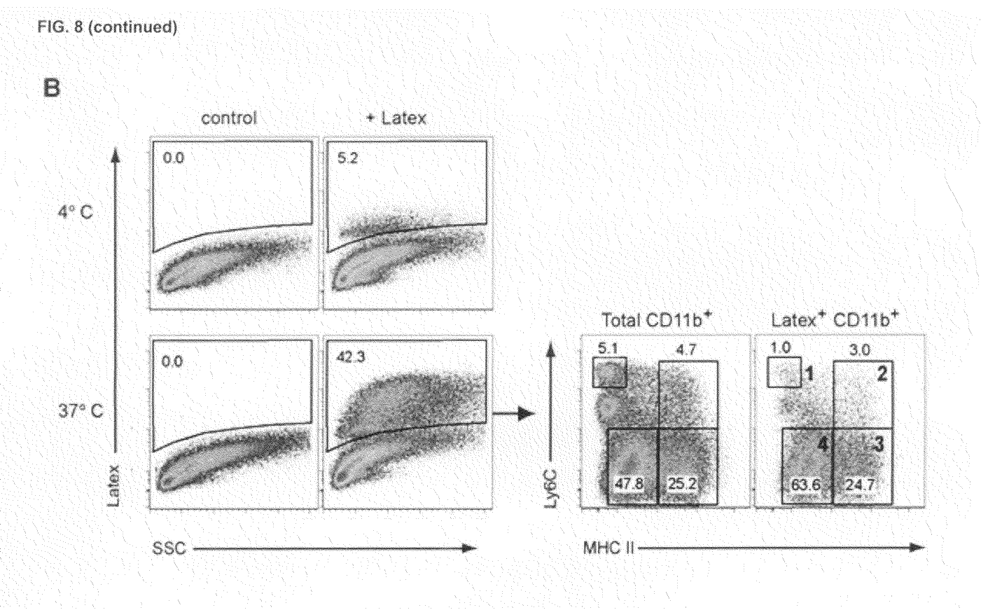

Next, ^{99m}Tc-labeled MMR Nanobodies and a control Nanobody recognizing a target not present in mice (the cAbBc1110 nanobody, raised against subunit 10 of the β-lactamase BcII enzyme of Bacillus cereus) were inoculated in TS/A tumor-bearing mice. Uptake of the MMR Nanobody in liver, spleen, lungs, kidneys and blood was similar as before (FIG. 12), whereas accumulation of the control Nanobody was below 1% IA/g for all organs except for lungs and kidneys. Interestingly, the MMR Nanobody showed significant accumulation in the subcutaneous TS/A tumor (>2.5% IA/g), whereas the uptake of the control Nanobody in the subcutaneous tumor had dropped below 0.5% IA/g at 1 hour 30 minutes post injection.

### Example 11

### TAM Targeting Using Anti-Cd206 Nb-Toxins

Anti-CD206 Nbs are covalently linked to a protein toxin for TAM cell killing. Candidate toxins are the diptheria-toxin or the Pseudomonas exotoxin. It is investigated whether Nb-toxin conjugates are able to induce TAM cell death both in vitro and in vivo. Next, the effect of Nb-toxin treatment on tumor growth is assessed. For this, different injection schemes and doses are evaluated, ideally obtaining tumor regression coupled to a low overall toxicity. Further, it is investigated whether in vivo TAM depletion results in reduced tumor angiogenesis. This is done by immunohistochemically counting the number of blood vessels in tumors of Nb-toxin treated or untreated mice.

Alternatively, TAM killing might alleviate immune suppression or induce an inflammatory environment favoring the development of anti-tumor immunity. Thereto, it is investigated whether Nb-toxin treatment expands tumor-infiltrating T cells (TILs). The activation of TILs is assessed by evaluating the expression of certain membrane markers and through intracellular measurement of cytokine production. CD8⁺ cytotoxic TILs are purified and their tumor killing potential is directly assessed in vitro. The impact of anti-tumor immunity is also evaluated by repeating the Nb-toxin treatment in Rag2^{-/-} or SCID mice, which do not have functional T or B cells.

### Example 12

### Targeting Tumors Using an Anti-Cd206 Nb-Enzyme/Prodrug Strategy

The observation that CD206 is expressed on TAMs from several independent tumor models, makes it a potential tumor-targeting marker for a variety of different cancers. CD206 is therefore an interesting candidate for developing antibody-directed enzyme prodrug therapies (ADEPT). In ADEPT an antibody is coupled to an enzyme which is able to convert a prodrug into a cytotoxic drug. We have previously proven that this also works with the Nb format.⁽²⁵⁾ Anti-CD206 Nbs can, for example, be coupled to β-lactamase, an enzyme which is able to release phenylenediamine mustard from the prodrug 7-(4-carboxybutanamido) cephalosporin mustard. Anti-CD206 Nb-enzyme conjugates can be injected in tumor-bearing mice, subsequently allowing clearance of unbound Nbs after which the prodrug is administered. This will result in a high toxicity at the tumor site, killing TAMs but also other bystander tumor cells, while having a low overall toxicity in the animal. We evaluate the efficacy of anti-CD206 Nb enzyme-prodrug therapies for inducing tumor regression in our preclinical tumor models.

### Example 13

### MMR as a Marker for the Differential Targeting of Tam Subsets In Vivo

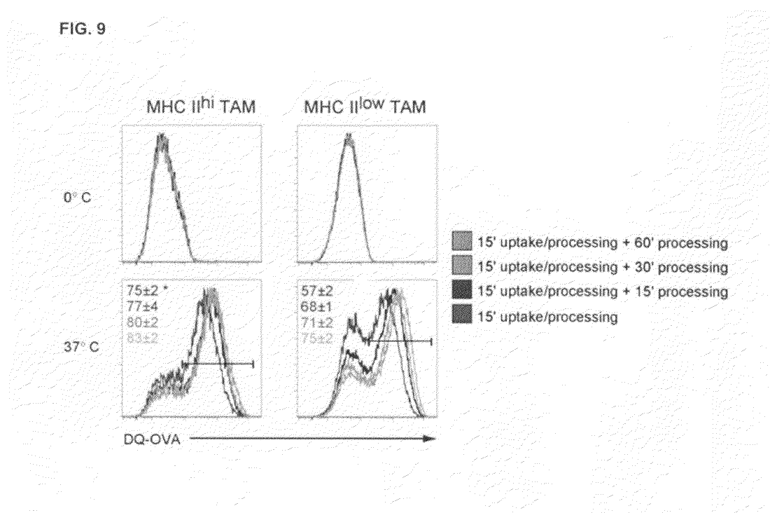

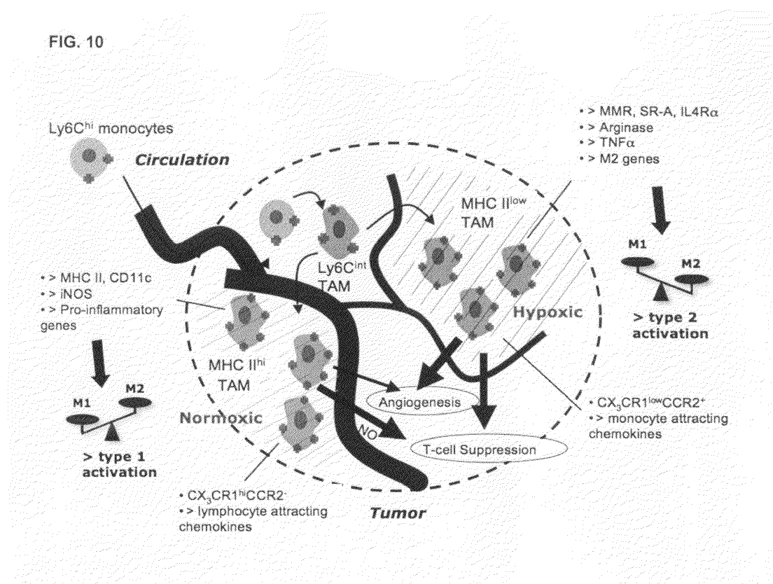

In the above Examples, it was shown that in tumor single cell suspensions, MMR was differentially expressed between MHC II^{hi} and MHC II^{low} TAMs, as assessed by flow cytometry using anti-MMR monoclonal antibodies. In addition, MMR was not/poorly expressed on CD11b⁻ cells, granulocytes, monocytes and Ly6C^{int} TAMs in the TS/A mouse mammary carcinoma model (FIG. 14). We next set out to investigate MMR expression patterns in tumor sections. TS/A mammary carcinoma sections were triple-stained for MMR (red), CD11b (blue) and MHC II (green) (FIG. 15). MMR and CD11b staining almost completely colocalized, showing that MMR⁺ cells were indeed TAMs. Interestingly however, MMR expression poorly colocalized with CD11b⁺MHC II⁺ cells (the majority corresponding to MHC II^{hi} TAMs), indicating that MMR staining was mainly restricted to MHC II^{low} TAMs. Therefore, MMR can be used for differentially labeling MHC II^{hi} and MHC II^{low} TAMs on tumor sections. Together with our flow cytometric results this indicates that MMR can be an interesting marker for specifically targeting MHC II^{low} TAMs in vivo.

### Example 14

### Generation of Anti-MMR Monovalent and Bivalent Nanobodies

Nanobodies (Nb) were raised against the recombinant extracellular portion of MMR (α-MMR Nb), as described in the Materials and Methods (see also Example 9; Table 4). The binding characteristics of the monovalent anti-MMR nanobodies were compared using surface Plasmon resonance measurements (Table 5). Nanobody clone 1 demonstrated an 8-fold higher apparent affinity compared to nanobody clone 3, and became hence the nanobody of choice for the remaining of this study. In addition, bivalent nanobodies were constructed by linking two anti-MMR nanobody 1 entities using (G₄S)₃ (GGGGSGGGGSGGGGS; SEQ ID NO:121), llama IgG2 hinge (AHHSEDPSSKAPKAPMA; SEQ ID NO:122) or human IgA hinge (SPSTPPTPSPSTPPAS SEQ ID NO:123) linkers. These bivalent anti-MMR molecules showed a 5-fold higher avidity compared to the monovalent clone 1 nanobody, which can be attributed largely to 3-fold increase in k_{d}. The different linkers used for bivalent nanobody construction did not seem to have a significant effect on the affinity of the molecules for the MMR antigen. As a negative control nanobody in all experiments, we consistently used α-BCII10 Nb, which is a binder of the β-lactamase BCII enzyme of Bacillus cereus.

### Example 15

### Ex Vivo Characterization of Anti-MMR Nanobodies

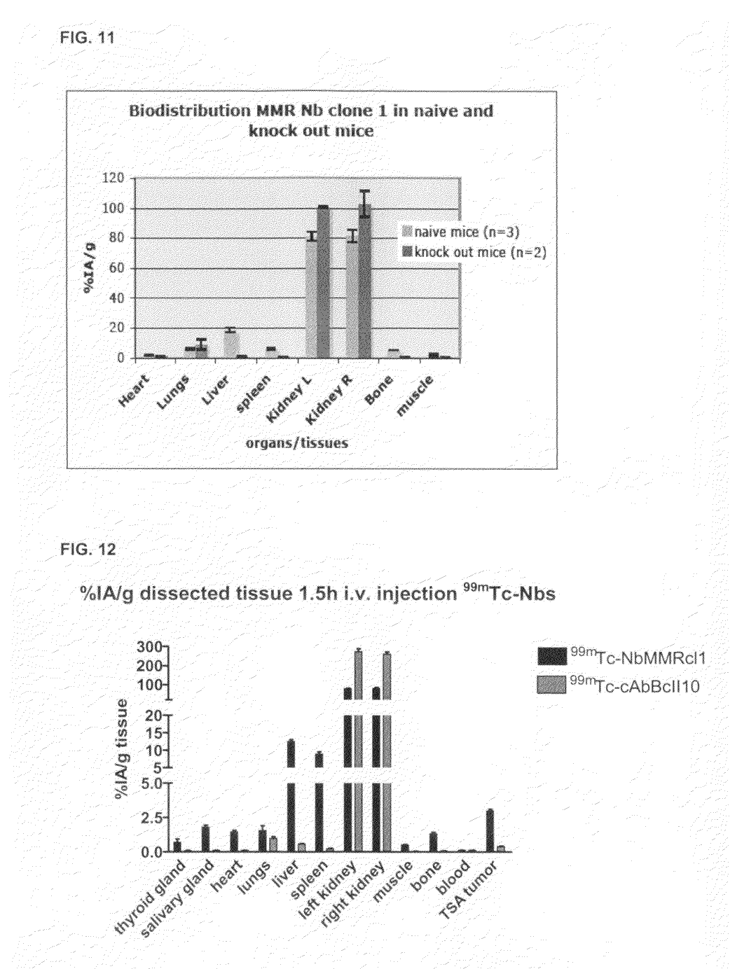

To investigate whether the anti-MMR Nb could bind to TAMs ex vivo, single cell suspensions were made of subcutaneous TS/A tumors and flow cytometric analyses were performed (FIG. 16). The anti-MMR Nb bound to a subset of CD11b⁺ cells, but not to CD11b⁻ cells (FIG. 16A). Within the CD11b⁺ fraction, anti-MMR Nb did not bind to monocytes (FIG. 16B, gate 1), granulocytes (Gate 5) and only very weakly to Ly6C^{int} TAMs (gate 2). Staining was therefore restricted to MHC II^{hi} (gate 3) and MHC II^{low} TAMs (gate 4), with the latter subset binding anti-MMR Nb to a much greater extent. These results are therefore in line with our previous observations using anti-MMR monoclonal antibodies. We conclude that in ex vivo tumor suspensions, the anti-MMR Nb stained mature TAMs and more intensely the MHC II^{low} subset.

### Example 16

### Assessment of the Biodistribution and Specificity of Anti-MMR Nanobody Clone 1 and its Bivalent Derivative in Naive Mice Using Pinhole SPECT/Micro-CT Analysis

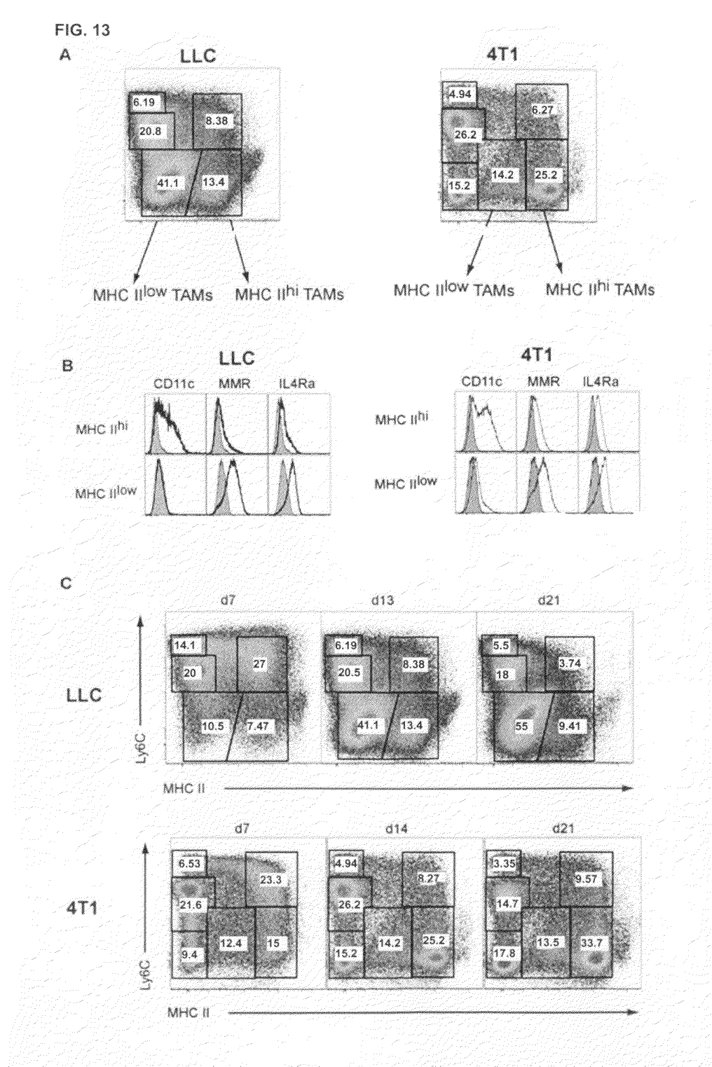

Next, we wished to assess whether the anti-MMR Nb clone 1 could be used for targeting and imaging of MMR-expressing cells in vivo. In first instance, this was investigated in naive mice. To this end, anti-MMR monovalent Nb were labeled with ^{99m}Tc and injected intravenously in naive C57BL/6 mice. 3 hours post injection, total-body scans were acquired using pinhole SPECT and micro-CT (FIG. 17), images were quantified and tracer uptake expressed as percentage injected activity per gram cubic centimeter (% IA/cm³) (Table 6). To ascertain the specificity of the anti-MMR Nb and to prove that any potential targeting was not due to aspecific retention, anti-MMR Nb were also injected in naive C57BL/6 MMR^{-/-} mice. In MMR^{-/-} mice, SPECT/micro-CT images show a high tracer uptake in the kidneys and urinary activity in the bladder, indicative of renal clearance, but only low background-level retention is seen in other organs (FIG. 17, Table 6). The only exception were the lungs, suggesting that lung-targeting was aspecific. In contrast, WT mice showed an increased retention of the anti-MMR Nb in several organs, including heart, bone, spleen and liver, with the latter two showing the most intense signals (FIG. 17). These results indicate that the anti-MMR monovalent Nb has a high in vivo specificity and can efficiently target organs such as the liver and spleen. A similar experiment was performed with the different bivalent anti-MMR Nb constructs, all of which showing an even increased uptake in the liver as compared to the monovalent molecule and a concomitant reduction in clearance via the kidneys (Table 7). Again, retention of bivalent anti-MMR Nb in all organs, except the lung, is MMR-specific and is absent in MMR^{-/-} mice. As was expected, retention of the control cAbBCII10 Nb is very low in all organs, resulting in a massive clearance via the kidneys (Table 7).

### Example 17

### Tumor-Targeting Potential and Specificity of Anti-MMR Nanobodies

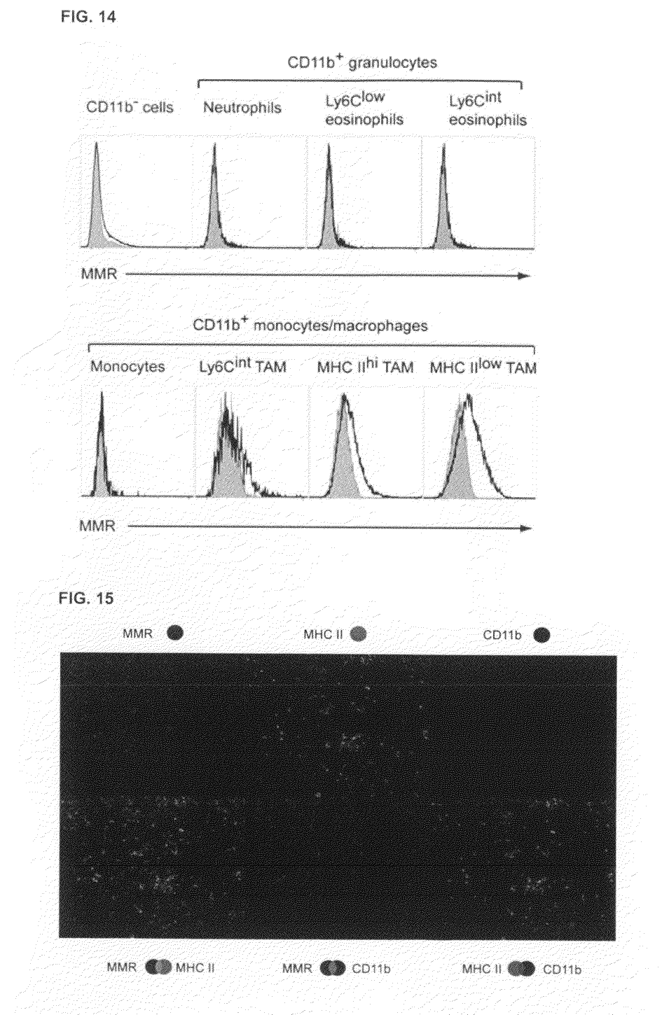

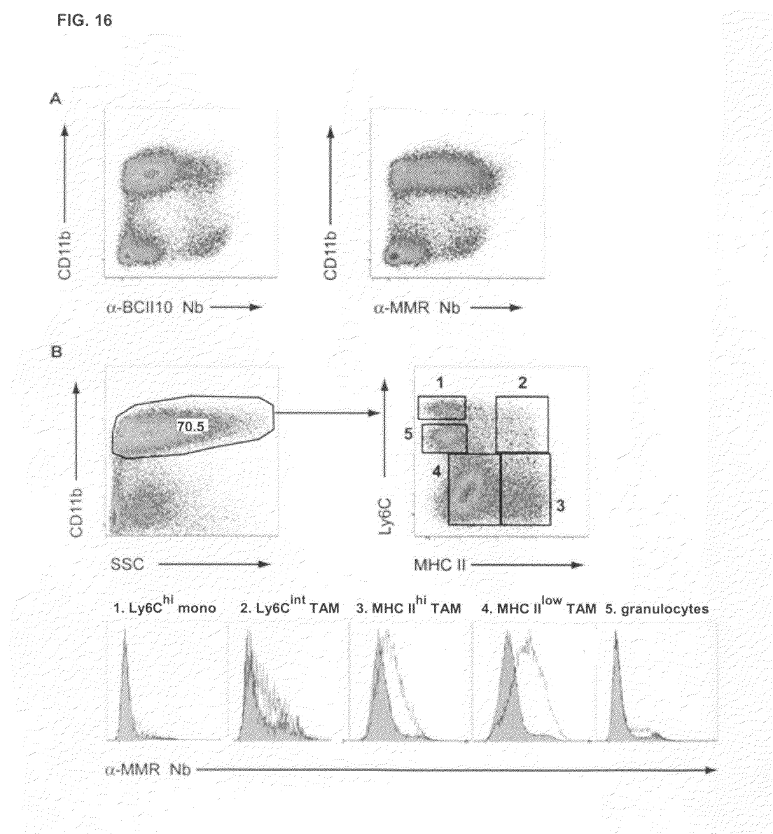

Next, we set out to investigate whether the anti-MMR Nb could be used to target TAMs in vivo. Hereto, ^{99m}Tc-labeled anti-MMR Nbs were injected intravenously in TS/A (Balb/c) and 3LL-R (C57BL/6) tumor-bearing mice and SPECT/micro-CT and ex vivo dissection analyses were performed. ^{99m}Tc-labeled cAbBCII10 Nbs were used as negative controls. In addition, to further ascertain the specificity of tumor uptake, 3LL-R tumors were also grown in C57BL/6 MMR^{-/-} mice. In these mice, 3LL-R tumors grew progressively and the distinct TAM subsets remained present as assessed by flow cytometry (data not shown). Interestingly, as observed by SPECT/micro-CT imaging, both TS/A and 3LL-R tumors showed a clear uptake of anti-MMR Nb, which was significantly higher than tumor uptake of cAbBCII10 Nb (FIG. 18, FIG. 19). These findings were confirmed through ex vivo dissection analysis, where the activity in the tumor and organs was assessed and expressed as injected activity per gram (% IA/g): TS/A tumor uptake was 3.02±0.10% IA/g for anti-MMR Nb and 0.40±0.03% IA/g for cAbBCII10 (Table 8); 3LL-R tumor uptake was 3.02±0.19% IA/g for anti-MMR Nb and 0.74±0.03% IA/g for cAbBCII10 (Table 9). Importantly, in 3LL-R tumor-bearing MMR^{-/-} mice, tumor uptake of anti-MMR Nb was reduced by 10-fold (0.33±0.03% IA/g, Table 9), showing that targeting in WT mice was receptor-specific.

### Example 18

### Blocking of Extratumoral Binding Sites by Excess Monovalent or Bivalent Anti-MMR Nb

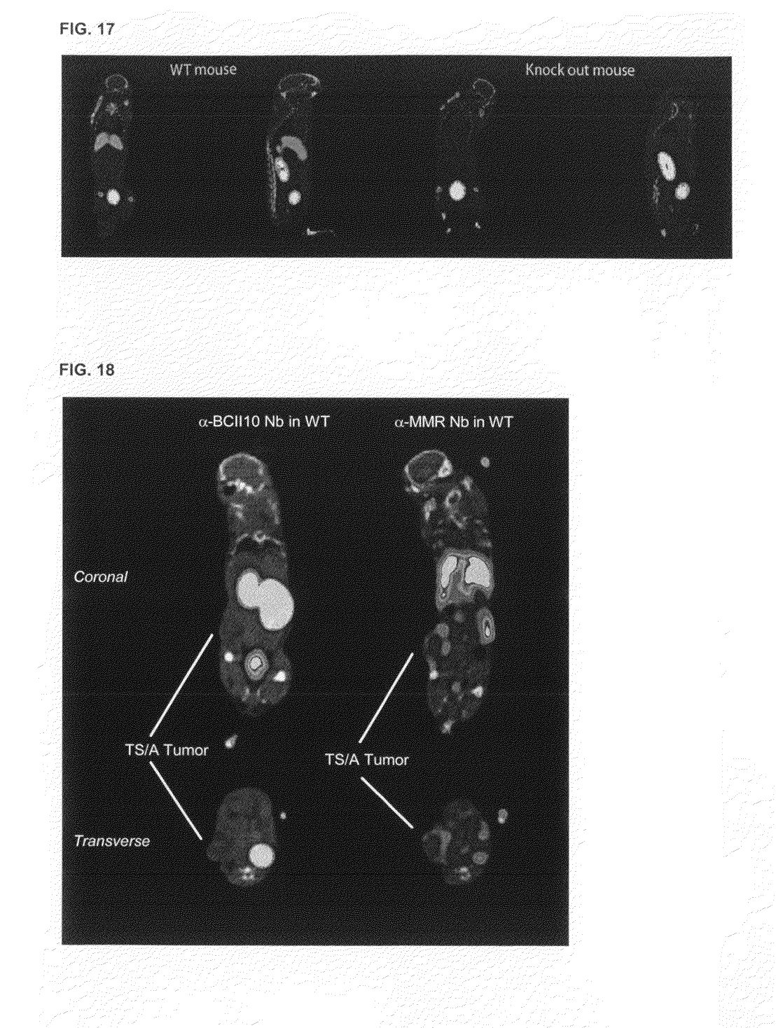

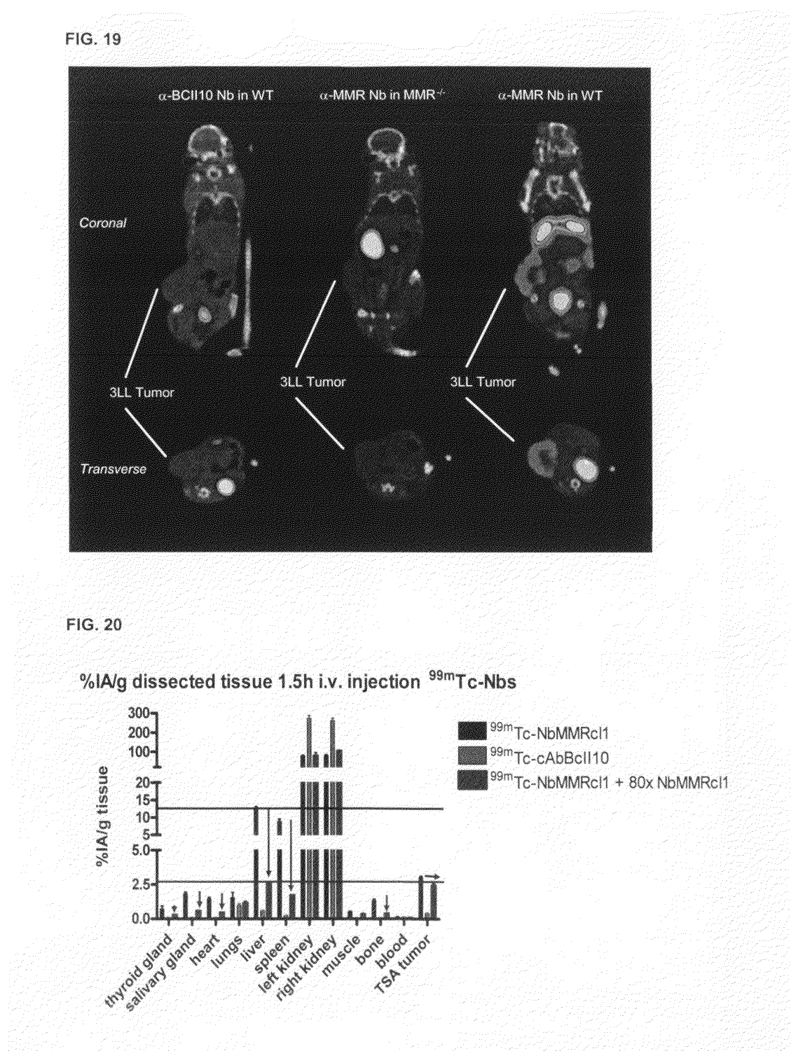

Both in the TS/A and 3LL-R model, ^{99m}Tc-labeled anti-MMR Nb accumulates to a higher extent in liver and spleen than in the tumor. Therefore, we sought for ways to minimize binding of labeled tracer in these extratumoral sites, while preserving tumor targeting. In first instance, we co-injected an 80-fold excess of cold unlabelled anti-MMR Nb and subsequently evaluated the biodistribution of ^{99m}Tc-labeled anti-MMR Nb. This strategy results in a strongly reduced accumulation of labeled Nb in all organs, except for the tumor, resulting in a similar level of specific uptake in tumor and liver (FIG. 20). Next, we hypothesized that the inherently enhanced biodistribution of bivalent anti-MMR Nb to the liver and its enhanced in vivo retention (lower clearance via the kidneys) could be exploited to block the extratumoral binding sites more efficiently. To this end, we co-injected ^{99m}Tc-labeled anti-MMR Nb with a 20-fold excess of cold bivalent anti-MMR and assessed the specific uptake of labeled Nb in distinct organs. Remarkably, while the retention of monovalent anti-MMR in all organs is reduced to the aspecific background level seen with the control Nb cAbBCII10, the uptake in tumors is only slightly diminished (FIG. 21). As a result, the specific uptake of labeled anti-MMR Nb is highest in the tumor.

### Example 19

### The Relative Abundance of TAM Subsets Correlates with Tumor Aggressiveness

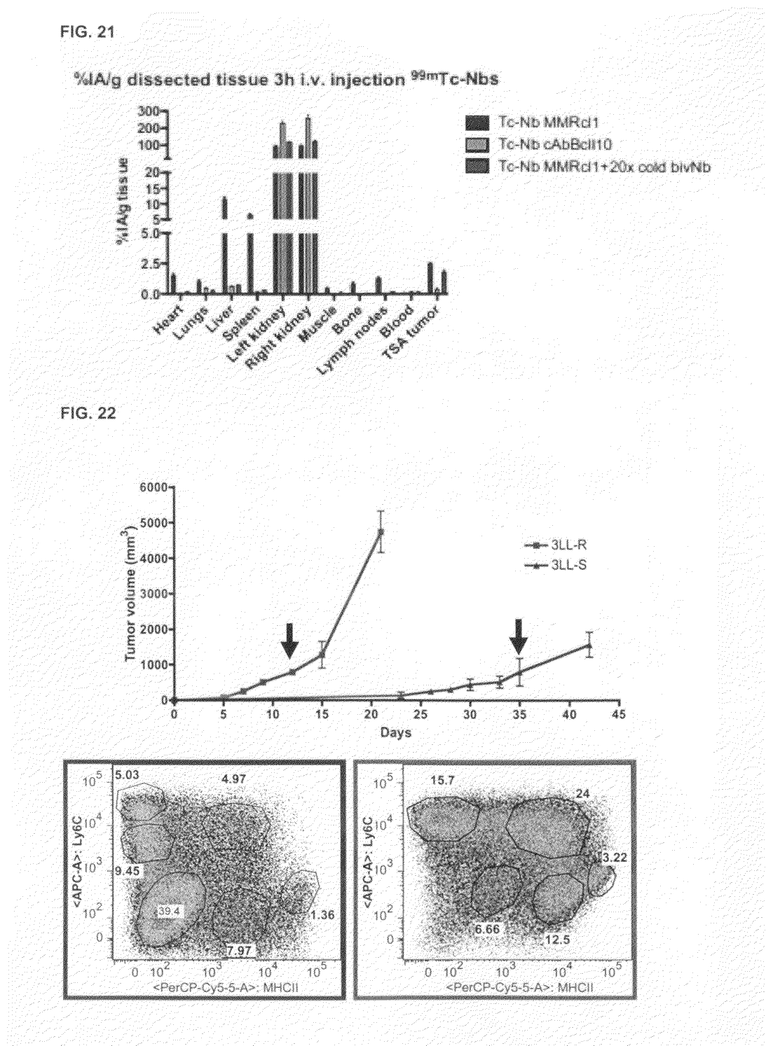

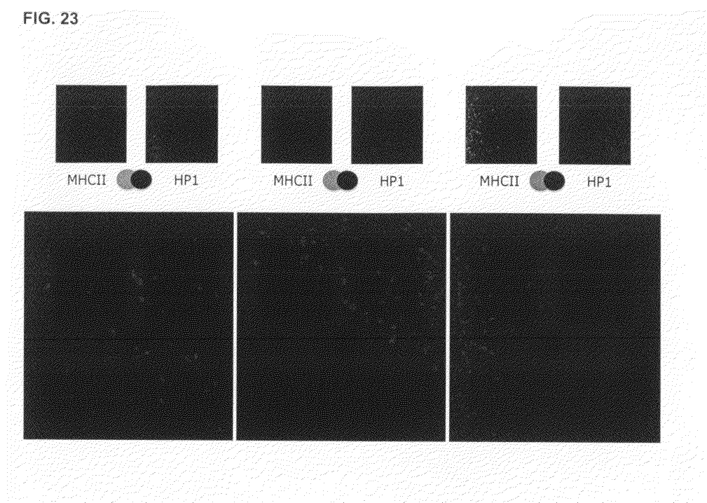

To assess whether the relative abundance of TAM subsets correlates with tumor aggressiveness, we injected high and low malignant 3LL lung carcinoma variants and evaluated the TAM subset distribution in the corresponding tumors. 3LL-R lung carcinoma cells establish rapidly growing tumors upon subcutaneous inoculation, reaching a tumor volume of about 1000 mm³ within 12 days (FIG. 22). In these tumors, the MHC II^{high} TAM subpopulation, which is located in normoxic regions, is outnumbered by the MHC II^{low} subset (FIG. 22, FIG. 23). In contrast, 3LL-S tumors grow much slower (1000 mm³ within about 35 days) and are dominated by the MHC II^{high} TAM subset (FIG. 22). A similar observation is made when comparing fast growing T241 fibrosarcoma tumors with slow growing T241-HRG tumors (data not shown). Together, these data indicate that the relative abundance of TAM subsets can be prognostic for tumor aggressiveness.

### Example 20

### Evaluation of the Anti-MMR-PE38 Immunotoxin

The anti-MMR Nb clone 1 was fused to the Pseudomonas exotoxin A as described in Materials and Methods, creating an MMR-specific immunotoxin. It was shown that the recombinant production of this immunotoxin results in a functional toxic moiety, with the ability to kill cancer (3LL-R, 3LL-S) and macrophage cell lines (J774) in vitro (data not shown). In vivo administration of the toxin does not result in lethality, even at the highest dose used (data not shown). Further, the ability of the immunotoxin to specifically eliminate MMR-positive cells in vivo is assessed, in particular MMR⁺MHC II^{low} TAM in tumors, and the consequences of TAM subset elimination for tumor characteristics (growth, metastasis, vessel density, vessel functionality, . . . ) is evaluated.

TABLE 1
Gene expression profile of MHC II^{hi} versus MHC II^{low} TAMS.

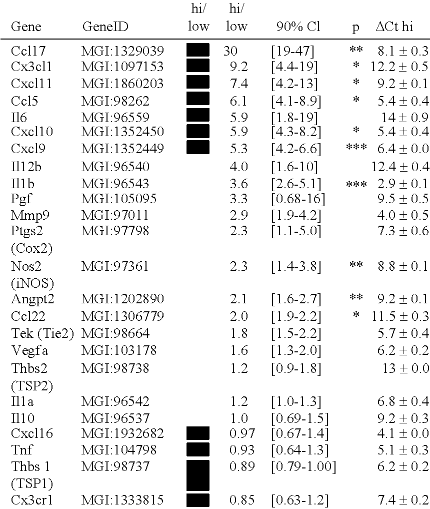

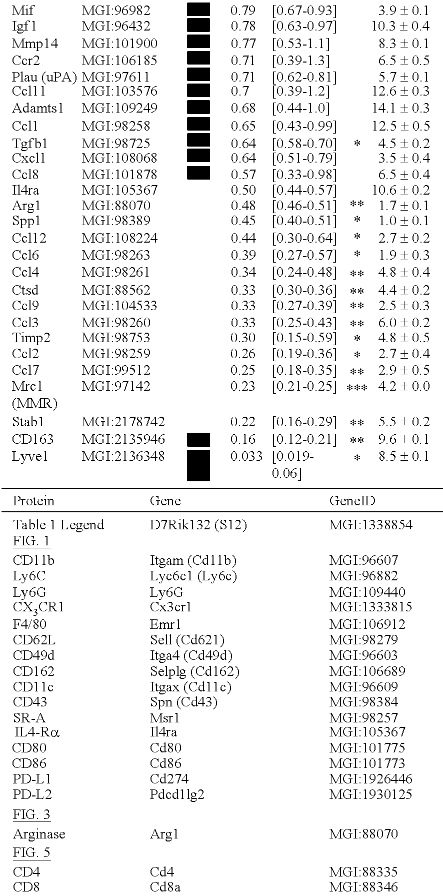

TAM subsets were sorted from 3 weeks tumor-bearing mice and their gene expression was
assesessed using aRT-PCR. The expression of each gene was normalized based on the S12
gene and is shown as the relative expression in MHC II^{hi} vs. MHC II^{low} TAMs (hi/low).
Values are the geometric means of 3-4 independent experiments and are color-coded
according to the level of induction. Accompanying 90% confidence intervals and p-values
are shown. * p <0.005; ** p <0.01; *** p <0.001. C_{T} represents the threshold cycle. The
ΔC_{T} was calculated for MHC II^{hi} TAMS and is defined as (C_{T}(gene)-C_{T}(S12)); values
represent mean ± SEM. Lower ΔCt corresponds to higher expression levels.

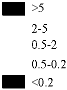

TABLE 2
List of commercial antibodies

| Markers | Clone | Manufacturer |
|---|---|---|
| CD11b PE-Cy7 | M1/70 | BD Bioscience |
| Ly6C AF647/AF488 | ER-MP20 | Serotec |
| Ly6G PE/FITC | 1A8 | BD Bioscience |
| IA/IE PE/FITC | M5/114.15.2 | BD Bioscience |
| IA/IE PercpCy5.5 | M5/114.15.2 | Serotec |
| IA/IE FITC | M5/114.15.2 | eBioscience |
| F4/80 PE/FITC | CI: A3-1 | Serotec |
| CCR3 FITC | 83101 | R&D Systems |
| CD62L PE | SK11 | BD Bioscience |
| CD11c PE | HL3 | BD Bioscience |
| CD43 PE | S7 | BD Bioscience |
| CD49d PE | 9C10(MFR4.B) | BD Bioscience |
| CD162 PE | 2PH1 | BD Bioscience |
| MMR FITC | MR5D3 | Serotec |
| SR-A PE | 2F8 | Serotec |
| IL4Rα | mIL4RM1 | BD Bioscience |
| Tie-2 PE | TEK4 | eBioscience |
| CD80 FITC | 16-10A1 | BD Bioscience |
| CD86 FITC | GL-1 | BD Bioscience |
| PD-L1/PE | MIH5 | eBioscience |
| PD-L2/PE | TY25 | eBioscience |
| anti-TNFα/APC | MP6-XT22 | BD Bioscience |
| Rabbit anti-iNOS | (M19) | Santa Cruz |
| anti-Rabbit/APC | polyclonal | Invitrogen |

TABLE 3
List of gene specific primers

| GENE | FORWARD PRIMER | REVERSE PRIMER |
|---|---|---|
| CCL17 | CCCATGAAGACCTTCACCTC (SEQ ID NO: 9) | CATCCCTGGAACACTCCACT (SEQ ID NO: 10) |
| CX3CL1 | ACTCCTTGATTGGTGGAAGC (SEQ ID NO: 11) | CAAAATGGCACAGACATTGG (SEQ ID NO: 12) |
| CXCL11 | TCCTTTCCCCAAATATCACG (SEQ ID NO: 13) | CAGCCATCCCTACCATTCAT (SEQ ID NO: 14) |
| CCL5 | GTGCCCACGTCAAGGAGTAT (SEQ ID NO: 15) | AGCAAGCAATGACAGGGAAG (SEQ ID NO: 16) |
| IL6 | GTCTTCTGGAGTACCATAGC (SEQ ID NO: 17) | GTCAGATACCTGACAACAGG (SEQ ID NO: 18) |
| CXCL10 | TCTGAGTCCTCGCTCAAGTG (SEQ ID NO: 19) | CCTTGGGAAGATGGTGGTTA (SEQ ID NO: 20) |
| CXCL9 | TCAACAAAAGAGCTGCCAAA (SEQ ID NO: 21) | GCAGAGGCCAGAAGAGAGAA (SEQ ID NO: 22) |
| IL12B | GAAAGACCCTGACCATCACT (SEQ ID NO: 23) | CCTTCTCTGCAGACAGAGAC (SEQ ID NO: 24) |
| IL1B | GTGTGGATCCAAAGCAATAC (SEQ ID NO: 25) | GTCTGCTCATTCATGACAAG (SEQ ID NO: 26) |
| PGF | GCACTGTGTGCCGATAAAGA (SEQ ID NO: 27) | TACCTCCGGGAAATGACATC (SEQ ID NO: 28) |
| MMP9 | TGAATCAGCTGGCTTTTGTG (SEQ ID NO: 29) | GTGGATAGCTCGGTGGTGTT (SEQ ID NO: 30) |
| PTGS2 (COX2) | CAGGCTGAACTTCGAAACAG (SEQ ID NO: 31) | CAGCTACGAAAACCCAATCA (SEQ ID NO: 32) |
| NOS2 | GCTTCTGGTCGATGTCATGAG (SEQ ID NO: 33) | TCCACCAGGAGATGTTGAAC (SEQ ID NO: 34) |
| ANGPT2 | GCATGTGGTCCTTCCAACTT (SEQ ID NO: 35) | GATCCTCAGCCACAACCTTC (SEQ ID NO: 36) |
| CCL22 | TGACTTGGGTCCTTGTCCTC (SEQ ID NO: 37) | AAGGAAGCCACCAATGACAC (SEQ ID NO: 38) |
| TEK (TIE2) | ACTTCGCAGGAGAACTGGAG (SEQ ID NO: 39) | AAGAAGCTGTTGGGAGGACA (SEQ ID NO: 40) |
| VEGFA | CAGGCTGCTGTAACGATGAA (SEQ ID NO: 41) | AATGCTTTCTCCGCTCTGAA (SEQ ID NO: 42) |
| THBS2 (TSP2) | GAAAGCATACCTGGCTGGAC (SEQ ID NO: 43) | ACAAAAGAGCCGTACCTGGA (SEQ ID NO: 44) |
| IL1A | TTTCAAAAGGAAGGGGACAA (SEQ ID NO: 45) | CCACCTAGAAAACCCTGCTG (SEQ ID NO: 46) |
| IL10 | ACTCAATACACACTGCAGGTG (SEQ ID N0: 47) | GGACTTTAAGGGTTACTTGG (SEQ ID NO: 48) |
| CXCL16 | GTCTCCTGCCTCCACTTTCT (SEQ ID NO: 49) | CTAAGGGCAGAGGGGCTATT (SEQ ID NO: 50) |
| TNF | CCTTCACAGAGCAATGACTC (SEQ ID NO: 51) | GTCTACTCCCAGGTTCTCTTC (SEQ ID NO: 52) |
| THBS1 (TSP1) | CGTTGCCATTGGAATAGAGA (SEQ ID NO: 53) | TGGCAAAGAGTCAAAACTGG (SEQ ID NO: 54) |
| CX3CR1 | CACCATTAGTCTGGGCGTCT (SEQ ID NO: 55) | GATGCGGAAGTAGCAAAAGC (SEQ ID NO: 56) |
| MIF | CTTTTAGCGGCACGAACGAT (SEQ ID NO: 57) | AAGAACAGCGGTGCAGGTAA (SEQ ID NO: 58) |
| IGF1 | TGACATGCCCAAGACTCAGA (SEQ ID NO: 59) | AGGTTGCTCAAGCAGCAAAG (SEQ ID NO: 60) |
| MMP14 | CCGGTACTACTGCTGCTCCT (SEQ ID NO: 61) | CACACACCGAGCTGTGAGAT (SEQ ID NO: 62) |
| CCR2 | CTCAGTTCATCCACGGCATA (SEQ ID NO: 63) | CAAGGCTCACCATCATCGTA (SEQ ID NO: 64) |
| PLAU (UPA) | TCTCCTGGGCAAGTGTAGGA (SEQ ID NO: 65) | GCCTGTGCAGAGTGAACAAA (SEQ ID NO: 66) |
| CCL11 | CTCCACAGCGCTTCTATTCC (SEQ ID NO: 67) | CTTCTTCTTGGGGTCAGCAC (SEQ ID NO: 68) |
| ADAMTS1 | CTGGGCAAGAAATCTGATGA (SEQ ID NO: 69) | TGGTTGTGGCAGGAAAGATA (SEQ ID NO: 70) |
| CCL1 | GGATGTTGACAGCAAGAGCA (SEQ ID NO: 71) | CTCATCTTCACCCCGGTTAG (SEQ ID NO: 72) |
| TGFB1 | CCAAGGAGACGGAATACAGG (SEQ ID N0: 73) | TCTCTGTGGAGCTGAAGCAA (SEQ ID NO: 74) |
| CXCL1 | TCATAGCCACACTCAAGAATG (SEQ ID NO: 75) | AAGCAGAACTGAACTACCATC (SEQ ID NO: 76) |
| CCL8 | TCTACGCAGTGCTTCTTTGC (SEQ ID NO: 77) | CCACTTCTGTGTGGGGTCTA (SEQ ID NO: 78) |
| IL4RA | GCAGATGGCTCATGTCTGAA (SEQ ID NO: 79) | CTCTGGGAAGCTGGGTGTAG (SEQ ID NO: 80) |
| ARG1 | TCACCTGAGCTTTGATGTCG (SEQ ID NO: 81) | TTATGGTTACCCTCCCGTTG (SEQ ID NO: 82) |
| SPP1 | GCTTGGCTTATGGACTGAGG (SEQ ID NO: 83) | CTTGTCCTTGTGGCTGTGAA (SEQ ID NO: 84) |
| CCL12 | GCCTCCTGCTCATAGCTACC (SEQ ID NO: 85) | GGGTCAGCACAGATCTCCTT (SEQ ID NO: 86) |
| CCL6 | ATGTCCAGCTTTGTGGGTTC (SEQ ID NO: 87) | AGGTCAGGTTCCGCAGATAA (SEQ ID NO: 88) |
| CCL4 | CCCACTTCCTGCTGTTTCTC (SEQ ID NO: 89) | GAGCAAGGACGCTTCTCAGT (SEQ ID NO: 90) |
| CTSD | CCTTCGCGATTATCAGAATCC (SEQ ID NO: 91) | TACTTATGGTGGACCCAGCA (SEQ ID NO: 92) |
| Ccl9 | CCAGATCACACATGCAACAG (SEQ ID NO: 93) | CTATAAAAATAAACACTTAGAGCCA (SEQ ID NO: 94) |
| Ccl3 | CGGAAGATTCCACGCCAATTC (SEQ ID NO: 95) | GGTGAGGAACGTGTCCTGAAG (SEQ ID NO: 96) |
| Timp2 | ATCGAACCCAGAGTGGAATG (SEQ ID NO: 97) | GCTAGAAACCCCAGCATGAG (SEQ ID NO: 98) |
| Ccl2 | CACTCACCTGCTGCTACTCATTCAC (SEQ ID NO: 99) | GGATTCACAGAGAGGGAAAAATGG (SEQ ID NO: 100) |
| Ccl7 | GACAAAGAAGGGCATGGAAG (SEQ ID NO: 101) | CATTCCTTAGGCGTGACCAT (SEQ ID NO: 102) |
| Mrc1 (MMR) | GCAAATGGAGCCGTCTGTGC (SEQ ID NO: 103) | CTCGTGGATCTCCGTGACAC (SEQ ID NO: 104) |
| Stab1 | ACGGGAAACTGCTTGATGTC (SEQ ID NO: 105) | ACTCAGCGTCATGTTGTCCA (SEQ ID NO: 106) |
| CD163 | GAGCATGAATGAAGTGTCCG (SEQ ID NO: 107) | TGCTGAAGTTGTCGTCACAC (SEQ ID NO: 108) |
| Lyve1 | CTGGCTGTTTGCTACGTGAA (SEQ ID NO: 109) | CATGAAACTTGCCTCGTGTG (SEQ ID NO: 110) |

TABLE 4
Anti-CD206 nanobody (anti-MMR nanobody clone 1 and 3)

| | |
|---|---|
| DNA seq + His tag | CAGGTGCAGCTGCAGGAGTCTGGAGGAGGCTTGGTGCAGCCTGGGGGGTCTCTGAGACTC |
| (clone 1) | TCCTGTGCAGCCTCTGGAAACATCTTCAGTATCAATGCCATCGGCTGGTACCGCCAGGCTCC |
| SEQ ID NO: 1 | AGGGAAGCAGCGCGAGTTGGTCGCAACTATTACTCTTAGTGGTAGCACAAACTATGCAGAC |
| | TCCGTGAAGGGCCGATTCTCCATCTCCAGAGACAACGCCAAGAACACGGTGTATCTGCAAA |
| | TGAACAGCCTGAAACCTGAGGACACGGCCGTCTATTACTGTAATGCTAACACCTATAGCGAC |
| | TCTGACGTTTATGGCTACTGGGGCCAGGGGACCCAGGTCACCGTCTCCTCACACCACCATCA |
| | CCATCAC |
| DNA seq - His tag | CAGGTGCAGCTGCAGGAGTCTGGAGGAGGCTTGGTGCAGCCTGGGGGGTCTCTGAGACTC |
| (clone 1) | TCCTGTGCAGCCTCTGGAAACATCTTCAGTATCAATGCCATCGGCTGGTACCGCCAGGCTCC |
| SEQ ID NO: 2 | AGGGAAGCAGCGCGAGTTGGTCGCAACTATTACTCTTAGTGGTAGCACAAACTATGCAGAC |
| | TCCGTGAAGGGCCGATTCTCCATCTCCAGAGACAACGCCAAGAACACGGTGTATCTGCAAA |
| | TGAACAGCCTGAAACCTGAGGACACGGCCGTCTATTACTGTAATGCTAACACCTATAGCGAC |
| | TCTGACGTTTATGGCTACTGGGGCCAGGGGACCCAGGTCACCGTCTCCTCA |
| Protein + His tag | QVQLQESGGGLVQPGGSLRLSCAASGNIFSINAIGWYRQAPGKQRELVATITLSGSTNYADSVK |
| (clone 1) | GRFSISRDNAKNTVYLQMNSLKPEDTAVYYCNANTYSDSDVYGYWGQGTQVTVSSHHHHHH |
| SEQ ID NO: 3 | |
| Protein - His tag | QVQLQESGGGLVQPGGSLRLSCAASGNIFSINAIGWYRQAPGKQRELVATITLSGSTNYADSVK |
| (clone 1) | GRFSISRDNAKNTVYLQMNSLKPEDTAVYYCNANTYSDSDVYGYWGQGTQVTVSS |
| SEQ ID NO: 4 | |
| DNA seq + His tag | CAGGTGCAGCTGCAGGAGTCTGGAGGAGGATTGGTGCAGGCTGGGGGCTCTCTGAGACTC |
| (clone 3) | TCCTGTGCAGCCTCTGGACGCACCTTCAGTAGAGATGCCATGGGCTGGTTCCGCCAGGCTCC |
| SEQ ID NO: 5 | AGGGAAGGAGCGTGAGTTTGTAGCAGGTATTAGCTGGAGTGGTGGTAGCACATACTATGC |
| | AGACTCCGTGAAGGGCCGATTCACCATCTCCAGGGACGGCGCCAAGAACACGGTAAATCTG |
| | CAAATGAACAGCCTGAAACCTGAGGACACGGCCGTTTATTACTGTGCAGCATCGTCGATTTA |
| | TGGGAGTGCGGTAGTAGATGGGCTGTATGACTACTGGGGCCAGGGGACCCAGGTCACCGT |
| | CTCCTCACACCACCATCACCATCAC |
| DNA seq - His tag | CAGGTGCAGCTGCAGGAGTCTGGAGGAGGATTGGTGCAGGCTGGGGGCTCTCTGAGACTC |
| (clone 3) | TCCTGTGCAGCCTCTGGACGCACCTTCAGTAGAGATGCCATGGGCTGGTTCCGCCAGGCTCC |
| SEQ ID NO: 6 | AGGGAAGGAGCGTGAGTTTGTAGCAGGTATTAGCTGGAGTGGTGGTAGCACATACTATGC |
| | AGACTCCGTGAAGGGCCGATTCACCATCTCCAGGGACGGCGCCAAGAACACGGTAAATCTG |
| | CAAATGAACAGCCTGAAACCTGAGGACACGGCCGTTTATTACTGTGCAGCATCGTCGATTTA |
| | TGGGAGTGCGGTAGTAGATGGGCTGTATGACTACTGGGGCCAGGGGACCCAGGTCACCGT |
| | CTCCTCA |
| Protein + His tag | QVQLQESGGGLVQAGGSLRLSCAASGRTFSRDAMGWFRQAPGKEREFVAGISWSGGSTYYAD |
| (clone 3) | SVKGRFTISRDGAKNTVNLQMNSLKPEDTAVYYCAASSIYGSAVVDGLYDYWGQGTQVTVSSH |
| SEQ ID NO: 7 | HHHHH |
| Protein - His tag | QVQLQESGGGLVQAGGSLRLSCAASGRTFSRDAMGWFRQAPGKEREFVAGISWSGGSTYYAD |
| (clone 3) | SVKGRFTISRDGAKNTVNLQMNSLKPEDTAVYYCAASSIYGSAVVDGLYDYWGQGTQVTVSS |
| SEQ ID NO: 8 | |
| DNA seq + His tag | CAGGTGCAGCTTCAGGAGTCTGGAGGAGGCTTGGTGCAGCCTGGGGGGTCTCTGAGACTC |
| (MMR biv IgA) | TCCTGTGCAGCCTCTGGAAACATCTTCAGTATCAATGCCATCGGCTGGTACCGCCAGGCTCC |
| SEQ ID NO: 111 | AGGGAAGCAGCGCGAGTTGGTCGCAACTATTACTCTTAGTGGTAGCACAAACTATGCAGAC |
| | TCCGTGAAGGGCCGATTCTCCATCTCCAGAGACAACGCCAAGAACACGGTGTATCTGCAAA |
| | TGAACAGCCTGAAACCTGAGGACACGGCCGTCTATTACTGTAATGCTAACACCTATAGCGAC |
| | TCTGACGTTTATGGCTACTGGGGCCAGGGGACCCAGGTCACCGTCTCCTCAAGCCCATCTAC |
| | ACCTCCCACACCATCACCATCCACACCACCGGCAAGTCAGGTGCAGCTGCAGGAGTCTGGA |
| | GGAGGCTTGGTGCAGCCTGGGGGGTCTCTGAGACTCTCCTGTGCAGCCTCTGGAAACATCT |
| | TCAGTATCAATGCCATCGGCTGGTACCGCCAGGCTCCAGGGAAGCAGCGCGAGTTGGTCGC |
| | AACTATTACTCTTAGTGGTAGCACAAACTATGCAGACTCCGTGAAGGGCCGATTCTCCATCT |
| | CCAGAGACAACGCCAAGAACACGGTGTATCTGCAAATGAACAGCCTGAAACCTGAGGACA |
| | CGGCCGTCTATTACTGTAATGCTAACACCTATAGCGACTCTGACGTTTATGGCTACTGGGGC |
| | CAGGGGACCCAGGTCACCGTCTCCTCACACCACCATCACCATCAC |
| Protein + His tag | QVQLQESGGGLVQPGGSLRLSCAASGNIFSINAIGWYRQAPGKQRELVATITLSGSTNYADSVK |
| (MMR biv IgA) | GRFSISRDNAKNTVYLQMNSLKPEDTAVYYCNANTYSDSDVYGYWGQGTQVTVSSSPSTPPTP |
| SEQ ID NO: 112 | SPSTPPASQVQLQESGGGLVQPGGSLRLSCAASGNIFSINAIGWYRQAPGKQRELVATITLSGST |
| | NYADSVKGRFSISRDNAKNTVYLQMNSLKPEDTAVYYCNANTYSDSDVYGYWGQGTQVTVSS |
| | HHHHHH |
| DNA seq + His tag | CAGGTGCAGCTTCAGGAGTCTGGAGGAGGCTTGGTGCAGCCTGGGGGGTCTCTGAGACTC |
| (MMR biv | TCCTGTGCAGCCTCTGGAAACATCTTCAGTATCAATGCCATCGGCTGGTACCGCCAGGCTCC |
| (Gly4Ser)3) | AGGGAAGCAGCGCGAGTTGGTCGCAACTATTACTCTTAGTGGTAGCACAAACTATGCAGAC |
| SEQ ID NO: 113 | TCCGTGAAGGGCCGATTCTCCATCTCCAGAGACAACGCCAAGAACACGGTGTATCTGCAAA |
| | TGAACAGCCTGAAACCTGAGGACACGGCCGTCTATTACTGTAATGCTAACACCTATAGCGAC |
| | TCTGACGTTTATGGCTACTGGGGCCAGGGGACCCAGGTCACCGTCTCCTCAGGCGGAGGCG |
| | GTAGTGGCGGAGGTGGATCTGGAGGCGGCGGTAGTCAGGTGCAGCTGCAGGAGTCTGGA |
| | GGAGGCTTGGTGCAGCCTGGGGGGTCTCTGAGACTCTCCTGTGCAGCCTCTGGAAACATCT |
| | TCAGTATCAATGCCATCGGCTGGTACCGCCAGGCTCCAGGGAAGCAGCGCGAGTTGGTCGC |
| | AACTATTACTCTTAGTGGTAGCACAAACTATGCAGACTCCGTGAAGGGCCGATTCTCCATCT |
| | CCAGAGACAACGCCAAGAACACGGTGTATCTGCAAATGAACAGCCTGAAACCTGAGGACA |
| | CGGCCGTCTATTACTGTAATGCTAACACCTATAGCGACTCTGACGTTTATGGCTACTGGGGC |
| | CAGGGGACCCAGGTCACCGTCTCCTCACACCACCATCACCATCAC |
| Protein + His tag | QVQLQESGGGLVQPGGSLRLSCAASGNIFSINAIGWYRQAPGKQRELVATITLSGSTNYADSVK |
| (MMR biv | GRFSISRDNAKNTVYLQMNSLKPEDTAVYYCNANTYSDSDVYGYWGQGTQVTVSSGGGGSGG |
| (GIy4Ser)3) | GGSGGGGSQVQLQESGGGLVQPGGSLRLSCAASGNIFSINAIGWYRQAPGKQRELVATITLSGS |
| SEQ ID NO: 114 | TNYADSVKGRFSISRDNAKNTVYLQMNSLKPEDTAVYYCNANTYSDSDVYGYWGQGTQVTVS |
| | SHHHHHH |
| DNA seq + His tag | CAGGTGCAGCTTCAGGAGTCTGGAGGAGGCTTGGTGCAGCCTGGGGGGTCTCTGAGACTC |
| (MMR biv g2c) | TCCTGTGCAGCCTCTGGAAACATCTTCAGTATCAATGCCATCGGCTGGTACCGCCAGGCTCC |
| SEQ ID N0: 115 | AGGGAAGCAGCGCGAGTTGGTCGCAACTATTACTCTTAGTGGTAGCACAAACTATGCAGAC |
| | TCCGTGAAGGGCCGATTCTCCATCTCCAGAGACAACGCCAAGAACACGGTGTATCTGCAAA |
| | TGAACAGCCTGAAACCTGAGGACACGGCCGTCTATTACTGTAATGCTAACACCTATAGCGAC |
| | TCTGACGTTTATGGCTACTGGGGCCAGGGGACCCAGGTCACCGTCTCCTCAGCGCACCACA |
| | GCGAAGACCCCAGCTCCAAAGCTCCCAAAGCTCCAATGGCACAGGTGCAGCTGCAGGAGTC |
| | TGGAGGAGGCTTGGTGCAGCCTGGGGGGTCTCTGAGACTCTCCTGTGCAGCCTCTGGAAAC |
| | ATCTTCAGTATCAATGCCATCGGCTGGTACCGCCAGGCTCCAGGGAAGCAGCGCGAGTMG |
| | TCGCAACTATTACTCTTAGTGGTAGCACAAACTATGCAGACTCCGTGAAGGGCCGATTCTCC |
| | ATCTCCAGAGACAACGCCAAGAACACGGTGTATCTGCAAATGAACAGCCTGAAACCTGAGG |
| | ACACGGCCGTCTATTACTGTAATGCTAACACCTATAGCGACTCTGACGTTTATGGCTACTGG |
| | GGCCAGGGGACCCAGGTCACCGTCTCCTCACACCACCATCACCATCAC |
| Protein + His tag | QVQLQESGGGLVQPGGSLRLSCAASGNIFSINAIGWYRQAPGKQRELVATITLSGSTNYADSVK |
| (MMR biv g2c) | GRFSISRDNAKNTVYLQMNSLKPEDTAVYYCNANTYSDSDVYGYWGQGTQVTVSSAHHSEDP |
| SEQ ID NO: 116 | SSKAPKAPMAQVQLQESGGGLVQPGGSLRLSCAASGNIFSINAIGWYRQAPGKQRELVATITLS |
| | GSTNYADSVKGRFSISRDNAKNTVYLQMNSLKPEDTAVYYCNANTYSDSDVYGYWGQGTQVT |
| | VSSHHHHHH |

TABLE 5
SPR kinetic and equilibrium parameters for anti-
MMR Nanobodies and bivalent Nanobody 1 derivatives.

| Sample | kₐ | SE (kₐ) | k_{d} | SE (k_{d}) | K_{D} | Chi² |
|---|---|---|---|---|---|---|
| Anti-MMR Nb1 | 5.76E+05 | 1.4E+3 | 0.01331 | 2.1E−5 | 2.31E−08 | 0.558 |
| Anti-MMR Nb3 | 9.73E+04 | 1.6E+2 | 0.01859 | 2.2E−5 | 1.91E−07 | 0.190 |
| biv MMR linker 1 GS | 1.04E+06 | 4.9E+3 | 0.004404 | 1.4E−5 | 4.22E−09 | 3.56 |
| biv MMR linker 2 g2c | 1.02E+06 | 4.8E+3 | 0.004107 | 1.4E−5 | 4.04E−09 | 2.50 |
| biv MMR linker 3 IgA | 9.13E+05 | 1.5E+4 | 0.004285 | 5.3E−5 | 4.69E−09 | 2.25 |

Nb: Nanobody; biv: bivalent; GS: (Gly₄Ser)₃ linker; g2c: Ilama IgG2 hinge linker; IgA: human IgA hinge linker; SE: standard error.

TABLE 6
Uptake values of ^{99m}Tc-labeled anti-MMR Nb clone 1 in
naive and MMR^{-/-} mice based on Pinhole SPECT/micro-CT
at 1 hour post injection.
Tracer uptake is expressed as percentage injected activity per
gram cubic centimeter (% IA/cm³).

| | | MMR Nb in MMR^{-/-} |
|---|---|---|
| Organs/Tissues | MMR Nb in WT (% IA/cm³) | (% IA/cm³) |
| Heart | 2.04 ± 0.21 | 1.13 ± 0.12 |
| Lungs | 5.96 ± 0.16 | 9.06 ± 2.43 |
| Liver | 18.66 ± 0.87 | 0.91 ± 0.16 |
| Spleen | 6.17 ± 0.31 | 0.34 ± 0.21 |
| Kidney Left | 80.98 ± 1.65 | 100.58 ± 0.4 |
| Kidney Right | 81.65 ± 2.32 | 102.82 ± 6.17 |
| Muscle | 1.74 ± 0.50 | 0.39 ± 0.22 |
| Bone | 5.02 ± 0.01 | 0.46 ± 0.02 |

TABLE 7
Uptake values of ^{99m}Tc-labeled bivalent anti-MMR Nb constructs (with (G₄S)₃, llama IgG2
hinge or human IgA hinge linkers), monovalent anti-MMR Nb clone 1, and control cAbBCII10 Nb in
naive and MMR^{-/-} mice based on Pinhole SPECT/micro-CT at 1 hour post injection.

| | | | Llama IgG2c | Llama IgG2c | Human IgA |
|---|---|---|---|---|---|
| Organs- | (G4S)3WT | (G4S)3MMR−/− | WT | MMR−/− | WT |
| Tissues | (% IA/cm3) | (% IA/cm3) | (% IA/cm3) | (% IA/cm3) | (% IA/cm3) |
| Heart | 1.549 ± 0.057 | 0.541 ± 0.013 | 1.416 ± 0.147 | 0.440 ± 0.070 | 1.395 ± 0.083 |
| Lungs | 1.053 ± 0.082 | 1.246 ± 0.038 | 0.987 ± 0.167 | 1.271 ± 0.130 | 0.936 ± 0.086 |
| Liver | 20.857 ± 0.215 | 0.930 ± 0.081 | 20.491 ± 0.578 | 1.658 ± 0.077 | 21.571 ± 0.435 |
| Spleen | 14.018 ± 1.669 | 0.634 ± 0.042 | 13.618 ± 1.497 | 1.347 ± 0.300 | 13.805 ± 1.353 |
| Kidney Left | 26.381 ± 2.054 | 225.129 ± 13.936 | 24.257 ± 1.129 | 193.162 ± 8.114 | 26.728 ± 3.014 |
| Kidney Right | 26.074 ± 2.227 | 212.682 ± 6.308 | 24.599 ± 2.053 | 202.343 ± 0.779 | 24.947 ± 2.463 |
| Muscle | 0.251 ± 0.034 | 0.224 ± 0.010 | 0.158 ± 0.023 | 0.216 ± 0.015 | 0.212 ± 0.045 |
| Bone | 1.466 ± 0.062 | 0.282 ± 0.016 | 1.041 ± 0.114 | 0.254 ± 0.030 | 1.089 ± 0.138 |

| | Human IgA | MMR Nb | cAbBCII10 |
|---|---|---|---|
| Organs- | MMR−/− | WT | WT |
| Tissues | (% IA/cm3) | (% IA/cm3) | (% IA/cm3) |
| Heart | 0.505 ± 0.057 | 2.793 ± 0.043 | 0.693 ± 0.128 |
| Lungs | 1.169 ± 0.161 | 2.543 ± 0.417 | 1.837 ± 0.271 |
| Liver | 1.176 ± 0.044 | 13.670 ± 0.741 | 2.637 ± 0.203 |
| Spleen | 0.477 ± 0.007 | 13.070 ± 0.251 | 0.933 ± 0.113 |
| Kidney Left | 210.760 ± 14.414 | 160.443 ± 13.153 | 415.643 ± 15.162 |
| Kidney Right | 214.144 ± 11.751 | 159.003 ± 13.700 | 408.597 ± 22.588 |
| Muscle | 0.205 ± 0.004 | ND | ND |
| Bone | 0.263 ± 0.022 | ND | ND |

Tracer uptake is expressed as percentage injected activity per gram cubic centimeter (% IA/cm³).

TABLE 8
Uptake values of ^{99m}Tc-labeled anti-MMR or cAbBCII10 Nb in TS/A
tumor-bearing WT mice, based on dissection at 3 hours post injection.
Tracer uptake is expressed as injected activity per gram tissue (% IA/g).

| | | cAbBcII10 Nb in WT |
|---|---|---|
| Organs/Tissues | anti-MMR Nb in WT (% IA/g) | (% IA/g) |
| Heart | 1.45 ± 0.12 | 0.10 ± 0.01 |
| Lungs | 1.55 ± 0.36 | 0.98 ± 0.12 |
| Liver | 12.60 ± 0.54 | 0.59 ± 0.02 |
| Spleen | 8.95 ± 0.60 | 0.24 ± 0.01 |
| Kidney Left | 79.67 ± 2.32 | 273.25 ± 14.76 |
| Kidney Right | 80.78 ± 3.62 | 261.16 ± 11.35 |
| Muscle | 0.52 ± 0.03 | 0.05 ± 0.01 |
| Bone | 1.33 ± 0.10 | 0.08 ± 0.01 |
| Blood | 0.13 ± 0.02 | 0.14 ± 0.01 |
| Tumor | 3.02 ± 0.10 | 0.40 ± 0.03 |

TABLE 9
Uptake values of ^{99m}Tc-labeled anti-MMR or cAbBCII10 Nb in 3LL
tumor-bearing WT or MMR^{-/-} mice, based on dissection
at 3 hours post injection.
Tracer uptake is expressed as injected activity per gram (% IA/g).

| | anti-MMR | anti-MMR Nb in | cAbBcII10 |
|---|---|---|---|
| | Nb in WT | MMR^{-/-} | Nb in WT |
| Organs/Tissues | (% IA/g) | (% IA/g) | (% IA/g) |
| Heart | 2.02 ± 0.11 | 0.06 ± 0.01 | 0.17 ± 0.01 |
| Lungs | 1.46 ± 0.05 | 1.02 ± 0.70 | 0.58 ± 0.04 |
| Liver | 9.55 ± 1.02 | 1.36 ± 1.06 | 1.03 ± 0.06 |
| Spleen | 4.61 ± 0.50 | 0.17 ± 0.02 | 0.41 ± 0.03 |
| Kidney Left | 108.61 ± 16.11 | 153.29 ± 27.22 | 368.79 ± 10.10 |
| Kidney Right | 88.60 ± 21.70 | 154.90 ± 20.71 | 305.21 ± 54.67 |
| Muscle | 0.61 ± 0.05 | 0.05 ± 0.02 | 0.08 ± 0.02 |
| Bone | 1.69 ± 0.10 | 0.06 ± 0.01 | 0.13 ± 0.01 |
| Blood | 0.10 ± 0.01 | 0.09 ± 0.01 | 0.24 ± 0.01 |
| Tumor | 3.02 ± 0.19 | 0.33 ± 0.03 | 0.74 ± 0.03 |

### REFERENCES

1. Mantovani A, Allavena P, Sica A, Balkwill F. Cancer-related inflammation. Nature 2008; 454:436-44.
2. Pollard J W. Tumour-educated macrophages promote tumour progression and metastasis. Nat Rev Cancer 2004; 4:71-8.
3. Lin E Y, Li J F, Gnatovskiy L, et al. Macrophages regulate the angiogenic switch in a mouse model of breast cancer. Cancer Res 2006; 66:1 1238-46.
4. Lewis C E, Pollard J W. Distinct role of macrophages in different tumor microenvironments. Cancer Res 2006; 66:605-12.
5. Varol C, Yona S, Jung S. Origins and tissue-context-dependent fates of blood monocytes. Immunol Cell Biol 2009; 87:30-8.
6. Auffray C, Sieweke M H, Geissmann F. Blood monocytes: development, heterogeneity, and relationship with dendritic cells. Annu Rev Immunol 2009; 27:669-92.
7. Martinez F O, Helming L, Gordon S. Alternative activation of macrophages: an immunologic functional perspective. Annu Rev Immunol 2009; 27:451-83.
8. Sica A, Schioppa T, Mantovani A, Allavena P. Tumour-associated macrophages are a distinct M2 polarised population promoting tumour progression: potential targets of anti-cancer therapy. Eur J Cancer 2006; 42:717-27.
9. Lewis C, Murdoch C. Macrophage responses to hypoxia: implications for tumor progression and anti-cancer therapies. Am J Pathol 2005; 167:627-35.
10. Nanni P, de Giovanni C, Lollini P L, Nicoletti G, Prodi G. TS/A: a new metastasizing cell line from a BALB/c spontaneous mammary adenocarcinoma. Clin Exp Metastasis 1983; 1:373-80.
11. Tacke F, Ginhoux F, Jakubzick C, van Rooijen N, Merad M, Randolph G J. Immature monocytes acquire antigens from other cells in the bone marrow and present them to T cells after maturing in the periphery. J Exp Med 2006; 203:583-97.
12. Tacke F, Alvarez D, Kaplan T J, et al. Monocyte subsets differentially employ CCR2, CCR5, and CX3CR1 to accumulate within atherosclerotic plaques. J Clin Invest 2007; 117:185-94.
13. Ojalvo L S, King W, Cox D, Pollard J W. High-density gene expression analysis of tumor-associated macrophages from mouse mammary tumors. Am J Pathol 2009; 174:1048-64.
14. Biswas S K, Gangi L, Paul S, et al. A distinct and unique transcriptional program expressed by tumor-associated macrophages (defective NF-kappaB and enhanced IRF-3/STAT1 activation). Blood 2006; 107:2112-22.
15. Hagemann T, Wilson J, Burke F, et al. Ovarian cancer cells polarize macrophages toward a tumor-associated phenotype. J Immunol 2006; 176:5023-32.
16. Hagemann T, Lawrence T, McNeish I, et al. “Re-educating” tumor-associated macrophages by targeting NF-kappaB. J Exp Med 2008; 205:1261-8.
17. Pugh C W, Ratcliffe P J. Regulation of angiogenesis by hypoxia: role of the HIF system. Nat Med 2003; 9:677-84.
18. Reis e Sousa C. Dendritic cells in a mature age. Nat Rev Immunol 2006; 6:476-83.
19. Tacke F, Ginhoux F, Jakubzick C, van Rooijen N, Merad M, Randolph G J. Immature monocytes acquire antigens from other cells in the bone marrow and present them to T cells after maturing in the periphery. J Exp Med. 2006; 203:583-597.
20. Tacke F, Alvarez D, Kaplan T J, et al. Monocyte subsets differentially employ CCR2, CCR5, and CX3CR1 to accumulate within atherosclerotic plaques. J Clin Invest. 2007; 1 17:185-194.
21. Van Rooijen N, Sanders A. Liposome mediated depletion of macrophages: mechanism of action, preparation of liposomes and applications. J Immunol Methods. 1994; 174:83-93.
22. Liu Y, Van Ginderachter J A, Brys L, De Baetselier P, Raes G, Geldhof A B. Nitric oxide-independent CTL suppression during tumor progression: association with arginase-producing (M2) myeloid cells. J. Immunol. 2003; 170:5064-5074.
23. Movahedi B, Gysemans C, Jacobs-Tulleneers-Thevissen D, Mathieu C, Pipeleers D. Pancreatic duct cells in human islet cell preparations are a source of angiogenic cytokines interleukin-8 and vascular endothelial growth factor. Diabetes. 2008; 57:2128-2136.
24. Movahedi K, Guilliams M, Van den Bossche J, et al. Identification of discrete tumor-induced myeloid-derived suppressor cell subpopulations with distinct T cell-suppressive activity. Blood. 2008; 111:4233-4244.
25. Cortez-Retamozo V, Backmann N, Senter P D, Wernery U, De Baetselier P, Muyldermans S and Revets H. Efficient cancer therapy with a nanobody-based conjugate. Cancer Res. 2004; 64:2853-2857.
26. Hamers-Casterman C., Atarhouch T., Muyldermans S., Robinson G., Hamers C., Songa E., Bendahman N. & Hamers R., 1993, Nature 363, p. 446-448.
27. Desmyter A., Transue T., Ghahroudi M., Dao-Thi M., Poortmans F., Hamers R., Muyldermans S. & Wyns L., 1996, Nat. Struct. Biol., p. 803-811.
28. Conrath, K., Lauwereys, M., Galleni, M., Matagne, A., Frere, J-M., Kinne, J., Wyns, L. and Muyldermans, S. (2001). β-Lactamase Inhibitors Derived from Single-Domain Antibody Fragments Elicited in the Camelidae Antimicrob Agents Chemother 45, 2807-2812
29. Saerens D., Ghassabeh G., Muyldermans S., 2008, Current Opinion in Pharmacology 8, p. 600-608.
30. Saerens D, Kinne J, Bosmans E, Wernery U, Muyldermans S, Conrath K. (2004). Single domain antibodies derived from dromedary lymph node and peripheral blood lymphocytes sensing conformational variants of prostate-specific antigen J Biol. Chem. 279: 51965-72.
31. Saerens D, Stijlemans B, Baral T N, Nguyen Thi G T, Wernery U, Magez S, De Baetselier P, Muyldermans S, Conrath K. J Parallel selection of multiple anti-infectome Nanobodies without access to purified antigens (2008) Immunol Methods. 329: 138-50.
32. Huarte E, Cubillos-Ruiz J R, Nesbeth Y C, Scarlett U K, Martinez D G, Buckanovich R J, Benencia F, Stan R V, Keler T, Sarobe P, Sentman C L, Conejo-Garcia J R. (2008) Depletion of dendritic cells delays ovarian cancer progression by boosting antitumor immunity. Cancer Res. 68: 7684-91.
33. Hwang J, Fitzgerald D J, Adhya S, Pastan I. (1987) Functional domains of Pseudomonas exotoxin identified by deletion analysis of the gene expressed in E. coli. Cell 48:129-36.
34. Harris N, Super M, Rits M, Chang G, Ezekowitz R A. (1992) Characterization of the murine macrophage mannose receptor: demonstration that the downregulation of receptor expression mediated by interferon-gamma occurs at the level of transcription. Blood 80: 2363-73.
35. Lefranc M P, Duprat E, Kaas Q, Tranne M, Thiriot A, Lefranc G. IMGT unique numbering for MHC groove G-DOMAIN and MHC superfamily (MhcSF) G-LIKE-DOMAIN. Dev Comp Immunol. 2005; 29(11):917-38.

What is claimed is:

1. A nanobody that specifically binds to a macrophage mannose receptor.

2. The nanobody of claim 1, wherein said nanobody specifically binds to the ectodomain of the macrophage mannose receptor.

3. The nanobody of claim 1, wherein said nanobody is fused to a moiety selected from the group consisting of toxin, a cytotoxic drug, an enzyme able to convert a prodrug into a cytotoxic drug, a radionuclide, and a cytotoxic cell.

4. The nanobody of claim 2, wherein said nanobody is fused to a moiety selected from the group consisting of toxin, a cytotoxic drug, an enzyme able to convert a prodrug into a cytotoxic drug, a radionuclide, and a cytotoxic cell.

5. The nanobody of claim 1 fused to a detectable label.

6. The nanobody of claim 2 fused to a detectable label.

7. A pharmaceutical composition comprising:
a therapeutically effective amount of the nanobody of claim 1, and
at least one of a pharmaceutically acceptable carrier, adjuvant, or diluent.

8. A pharmaceutical composition comprising:
a therapeutically effective amount of the nanobody of claim 2, and
at least one of a pharmaceutically acceptable carrier, adjuvant, or diluent.

9. A pharmaceutical composition comprising:
a therapeutically effective amount of the nanobody of claim 3, and
at least one of a pharmaceutically acceptable carrier, adjuvant, or diluent.

10. A pharmaceutical composition comprising:
a therapeutically effective amount of the nanobody of claim 4, and
at least one of a pharmaceutically acceptable carrier, adjuvant, or diluent.

11. A pharmaceutical composition comprising:
a therapeutically effective amount of the nanobody of claim 5, and
at least one of a pharmaceutically acceptable carrier, adjuvant, or diluent.

12. A pharmaceutical composition comprising:
a therapeutically effective amount of the nanobody of claim 6, and
at least one of a pharmaceutically acceptable carrier, adjuvant, or diluent.

13. A method of inhibiting tumor growth or tumor metastases in a mammal in need thereof, the method comprising:
selectively targeting TAM subpopulations linked to different intratumoral regions, a hypoxic region, or a normoxic region of a solid tumor.

14. The method according to claim 13, wherein the TAM subpopulation is defined as MHC II^{low}.

15. The method according to claim 13, comprising:
administering to the mammal a pharmaceutically effective amount of a nanobody that specifically binds to a macrophage mannose receptor.

16. A method of preventing and/or treating cancer in a mammal, the method comprising:
administering a pharmaceutically effective amount of a nanobody that specifically binds to a macrophage mannose receptor a mammal in need thereof.

17. A method of imaging tumor cells in a mammal suffering from or suspected to suffer from cancer comprising selectively visualizing TAM subpopulations linked to different intratumoral regions, such as hypoxic or normoxic regions of a solid tumor.

18. The method of claim 17, wherein the TAM subpopulation is defined as MHC II^{low}.

19. The method of claim 17, comprising administering to the mammal a nanobody that specifically binds to a macrophage mannose receptor fused to a detectable label.

20. A method of diagnosing cancer or prognosing cancer aggressiveness in a subject, the method comprising:
determining the relative percentage of TAM subpopulations.
